(19) United States
(12) Patent Application Publication
(10) Pub. No.: US 2012/0064002 A1
(43) Pub. Date: Mar. 15, 2012

(54) Enantiomer-pure (4S,8S)- and (4R,8R)-4-p-Nitrobenzyl-8-methyl-3,6,9-triaza-3N,6N,9N-tricarboxymethyl-1,11-undecanedioic Acid and Derivatives Thereof, Process for their Production and Use for the Production of Pharmaceutical Agents

(76) Inventors: Lutz LEHMANN, Berlin (DE); Matthias FRIEBE, Berlin (DE); Thomas BRUMBY, Berlin (DE); Detlev SUELZLE, Berlin (DE); Johannes PLATZEK, Berlin (DE)

(21) Appl. No.: 13/076,837

(22) Filed: Mar. 31, 2011

Related U.S. Application Data
(63) Continuation of application No. 12/845,174, filed on Jul. 28, 2010, now abandoned.
Application No. 12/845,174 is a continuation of application No. 10/771,149, filed on Feb. 4, 2004, now abandoned.
(60) Provisional application No. 60/446,538, filed on Feb. 12, 2003.

(30) Foreign Application Priority Data
Feb. 4, 2003 (DE) 10305463.4

(51) Int. Cl. A61K 51/00; C07C 229/28; C07C 331/28; C07K 16/00

(52) U.S. Cl. 424/1.61; 530/391.5; 560/37; 558/17

(57) ABSTRACT

Enantiomer-pure compounds of general formulas VIIa and VIIb

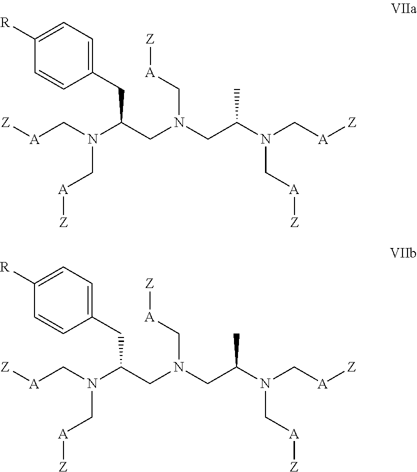

in which

A stands for a group —COO—, and Z and R have different meanings, as well as use thereof are described.

This application claims the benefit of the filing date of U.S. Provisional Application Ser. No. 60/446,538 filed Feb. 12, 2003.

The invention relates to the subjects that are characterized in the claims, i.e., (4S,8S)- and (4R,8R)-4-p-nitrobenzyl-8-methyl-3,6,9-triaza-³N,⁶N,⁹N-tricarboxymethyl-1,11-undecanedioic acid and derivatives thereof, process for their production and their use for the production of pharmaceutical agents for radiodiagnosis, radiotherapy or NMR diagnosis.

The use of radiopharmaceutical agents for diagnostic and therapeutic purposes has been known for a long time in the area of biological and medical research. In particular, radiopharmaceutical agents are used in this connection to visualize certain structures, such as, for example, the skeleton, organs or tissue. The diagnostic application requires the use of those radioactive agents that accumulate after administration specifically in the structures in patients who are to be examined. These locally accumulating radioactive agents can then be traced, recorded or scintigraphed by means of suitable detectors, such as, for example, scintillation cameras or other suitable recording processes. The distribution and relative intensity of the detected radioactive agent characterizes the site of a structure in which the radioactive agent is found and can visualize the presence of anomalies in structures and functions, pathological changes, etc.

In a similar way, radiopharmaceutical agents can be administered to patients as therapeutic agents to irradiate certain pathological tissues or areas. Such treatment requires the production of radioactive therapeutic agents that accumulate in certain structures, organs or tissues.

Zevalin® represents a radiopharmaceutical agent developed by the company IDEC Pharmaceuticals Corp. for treatment of non-Hodgkins-lymphoma (see, e.g., Cancer (2002) Feb. 15; 94, (4 Suppl):1349-57). In this connection, radiating ions are β-emitting ⁹⁰Y, which are bonded by a chelating agent (methyl-substituted-diethylene-triamine-pentaacetic acid derivative (MX-DTPA)) to a tumor-specific antibody.

The nuclear magnetic resonance (NMR) is now a broadly used method of medical diagnosis that is employed for in-vivo imaging, with which bodily vessels and bodily tissue (including tumors) can be visualized with the measurement of magnetic properties of protons in bodily water. For this purpose, e.g., contrast media are used that produce a contrast enhancement in the resulting images by influencing certain NMR parameters of the body protons (e.g., the relaxation times T¹ and T²) or make these images readable only. Primarily complexes of paramagnetic ions, such as, e.g., gadolinium-containing complexes (e.g., Magnevist®), are used based on the effect of the paramagnetic ions on the shortening of the relaxation times.

Both paramagnetic ions, such as, e.g.: Gd³⁺, Mn²⁺, Cr³⁺, Fe³⁺ and Cu²⁺, and many metallic radionuclides cannot be administered in free form as solutions, since they are highly toxic. To make these ions suitable for in-vivo use, they are generally complexed. For example, in EP-A-0 071 564, i.a., the meglumine salt of the gadolinium(III) complex of the diethylenetriaminepentaacetic acid (DTPA) is described as a contrast medium for NMR tomography. A preparation that contains this complex was accepted worldwide as the first NMR contrast medium under the name Magnevist®. This contrast medium is dispersed extracellularly after intravenous administration and is eliminated renally by glomerular secretion. A passage of intact cell membranes is virtually not observed. Magnevist® is especially well suited for the visualization of pathological areas (e.g., inflammations, tumors).

The known radiotherapeutic agents and contrast media, however, cannot be used satisfactorily for all applications. Many of these agents are thus dispersed in the entire extracellular space of the body. To increase the efficiency of these agents in in-vivo diagnosis and therapy, an attempt is made to increase their specificity and selectivity, for example, in target cells or desired areas and structures of the body. An improvement of these properties can be achieved, for example, by coupling the metal complexes to biomolecules according to the “Drug-Targeting” principle. Plasma proteins, antibodies, their fragments, hormones, growth factors and substrates of receptors and enzymes (e.g., WO 97/12850, Institut für Diagnostikforschung an der FU [Institute for Diagnostic Research of the Free University] Berlin) may be biomolecules. Up until now, however, e.g., the tumor specificity (tumor concentration) is not yet high enough in many cases, which is an important goal especially in radioimmunotherapy.

In addition, it is desirable to make available agents for diagnosis and therapy that in addition to as high a target specificity as possible have a high in-vivo stability for complexed metal ions that are toxic in most cases.

One object of the invention was therefore to make available new agents for radiodiagnosis and NMR diagnosis as well as radiotherapy that do not have the above-mentioned drawbacks and that do have in particular a high in-vivo stability, good compatibility and primarily organ-specific properties. On the one hand, the retention in the tumor tissues or organs to be examined should be sufficient to achieve at a low dosage the quality of images or adequate irradiation necessary for an efficient diagnosis and therapy. On the other hand, however, as quick as possible and as largely complete an excretion of metals from the body is to be ensured. Also, the NMR contrast media are to show a high proton relaxivity and thus allow a reduction of the dose in the case of an increase in signal intensity.

Various tests were undertaken to improve the properties of DTPA derivatives that can be biocoupled by the introduction of substituents.

Brechbiel et al. describe, e.g., a detailed synthesis of methyl-substituted DTPA derivatives, which can be coupled, for example, to antibodies (“A Convenient Synthesis of Bifunctional Chelating Agents Based on Diethylenetriaminepentaacetic Acid and Their Coordination Chemistry with Yttrium,” Bionconjugate Chemistry, (1991), 180-186. “Synthesis of (1-(p-Isothiocyanatobenzyl) Derivatives of DTPA and EDTA. Antibody Labeling and Tumor-Imaging Studies.” Inorg. Chem. (1986), 25, 2772-2781.). In Patent Application WO 88/01618 by Gansow et al., DTPA, which is provided with a methyl substituent in 8-position and a para-functionalized benzyl substituent in 4-position, is disclosed.

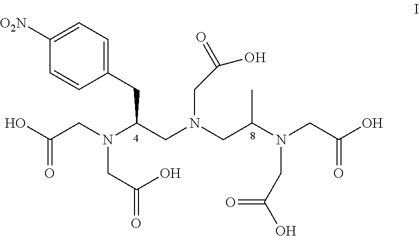

Compound I was named MX-DTPA, mx-DTPA or else 1B4M-H₅DTPA.

Patent Application W001/41743 of the company IDEC Pharmaceuticals Corporation describes a regioselective synthesis of compound I that starts from Boc-protected (S)-p-nitrophenylalanine and a mono-protected diamine. The stereogenic center in 4-position of this compound is described as S-configured; the stereogenic center in 8-position is not defined.

As already mentioned above, MX-DTPA is a component of the preparation ZEVALIN® for the treatment of non-Hodgkin's lymphoma. Also here, the mixture that consists of (4S,8R)- and (4S,8S)-MX-DTPA is used as a chelating ligand.

McMurry et al. (J. Med. Chem., (1998), 41, 3546-3549) were able to show that cyclohexyl-substituted DTPA, which are substituted with nitrobenzyl, have a preferred configuration because of the rigid and bulky structure of the cyclohexane ring. Compound II thus exhibits a higher in-vitro and in-vivo stability than compound III.

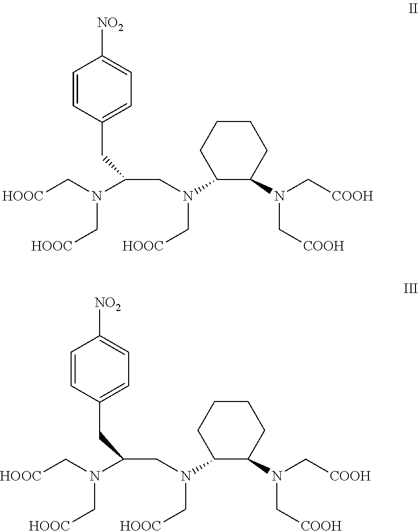

Although MX-DTPA (I) showed well-studied and acceptable properties—e.g., increased complex stability compared to the unsubstituted DTPA, it nevertheless remains desirable to further increase the metal complex stability in vivo and to make available agents that have as high a reliability as possible for diagnosis and therapy.

It has now been found that MX-DTPA derivatives, in which the comparatively small methyl substituent (8-position) had been introduced enantioselectively, with suitable configuration exhibits, surprisingly enough, a considerably higher thermodynamic stability in vitro that the diastereomer mixture (IV) (see below).

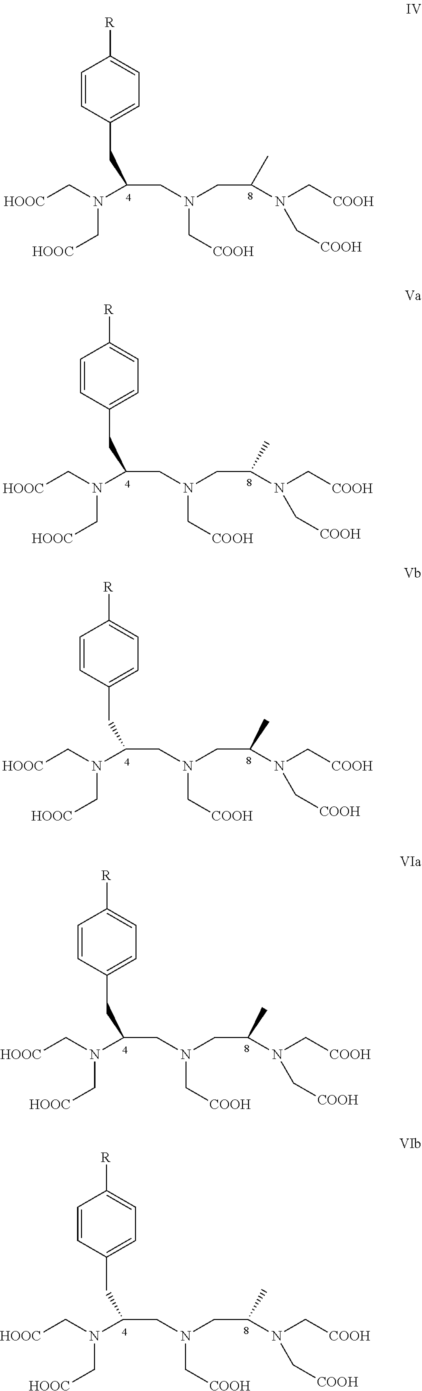

In this case, as was discovered, the configuration of methyl- and benzyl substituent must be present as (4S,8S) (see compound Va) or (4R,8R) (see compound Vb) to achieve the described positive effect. The configurations (4S,8R) or (4R,8S) (see compound VI a or b), however, result in reduced complex stability. By way of example, an impressive experiment could show that in a mixture of one equivalent cach of Va (R═—NO2), VIa (R═—NO2) and Gd(III) salt, almost exclusively the Gd complex of compound Va is formed. In addition, the thermodynamic stability constant fot compound Va with log K_{Y}=24.7±0.7, which thus is considerably increased compared to the stability constant of the diastereomer mixture IV (log K_{Y}=22.5 (J. Med. Chem. (1998), 41,3546-3549)), could be determined. It was found, moreover, that the radiotoxicity of the metal complex of compound Va is reduced in vivo compared to compound VIa.

This result is unexpected and surprising, since methyl substituents in 8-position are not rigid “space-organizing” or sterically exacting structures as is the case in cyclohexyl substituents of compounds II and III. It would thus be expected that all four stereoisomers would exhibit almost identical complexing properties. As already described, however, it was shown that in comparison, compounds Va and Vb according to the invention have considerably better complexing properties than compounds VIa and VIb.

Moreover, the compounds according to the invention show a good relaxivity and good water solubility, such that they are suitable as pharmaceutical agents especially for radiodiagnosis and NMR diagnosis as well as radiotherapy.

The invention thus relates to compounds of general formulas VIIa and VIIb

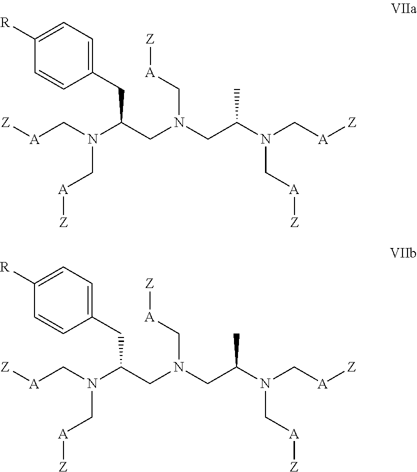

in which
Z stands for a hydrogen atom or a metal ion equivalent of an element of atomic numbers 21-29, 31, 32, 37-39, 42-44, 46, 47, 49, 58-71, 75, 77, 82 or 83,
A stands for a group —COO—,
R stands for a nitro group, an amino group, or another functional group, which can be linked with a biomolecule, or for a straight-chain or branched, saturated or unsaturated C₁-C₂₅-alkyl radical that is optionally interrupted by one to six 0 atoms or phenylene,
—NHCO—, —CONH—,

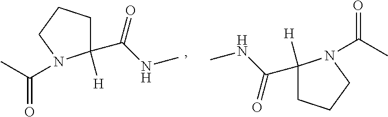

and/or —NH—(C═S)—NH— groups and that optionally is substituted at any location with one to six carboxyl groups, hydroxyl groups, amino groups or other functional groups, as well as their salts with organic or inorganic bases, provided that the alkyl radical contains at least one functional group that can be linked with a biomolecule and that at least two Z stand for a metal ion equivalent.

Radical R can stand for an alkyl radical with 1-25 carbon atoms (whereby it contains at least one functional group). This alkyl radical can be straight-chain or branched, saturated or unsaturated (e.g.:

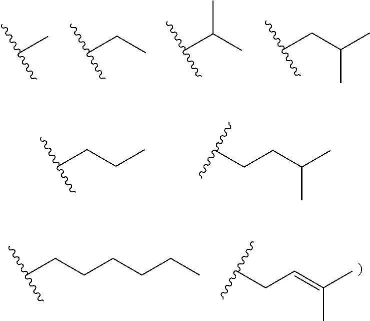

and is provided at any location with one to six carboxyl-, hydroxyl-, and/or amino groups (e.g.:

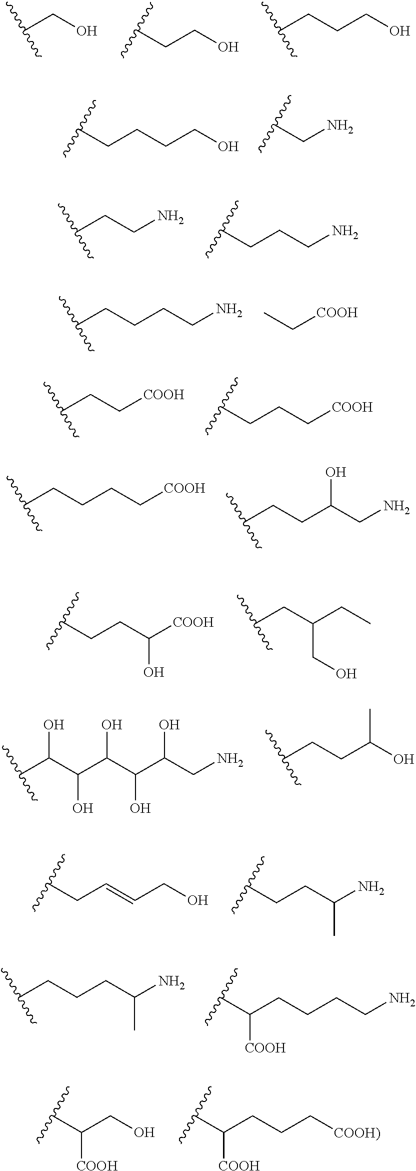

or at least one other functional binding group, which can be linked with a biomolecule—such as, e.g., carboxyl, activated carboxyl, amino, nitro, isocyanate, isothiocyanate, hydrazine, semicarbazide, thiosemibarbazide, chloroacetamide, bromoacetamide, iodoacetamide, acryl, acylamino, mixed anhydrides, azide, acid chloride, hydroxide, sulfonyl chloride, vinyl sulfone, carbodiimide, maleimide, dioxo or another functional binding group (e.g.,

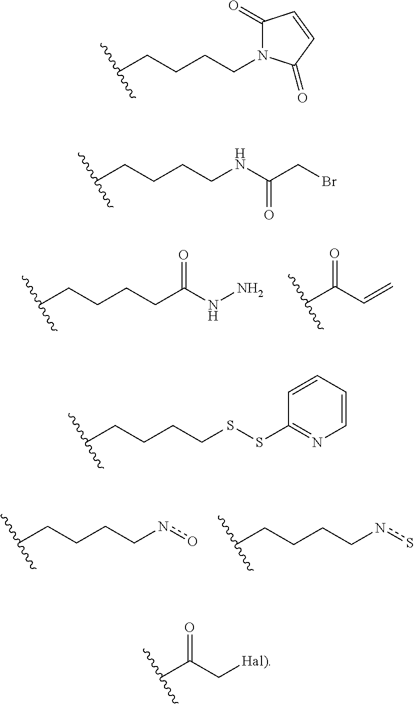

The C₁-C₂₅ alkyl radical optionally can be interrupted by one to six O atoms, phenylene,

### —NHCO—, —CONH—, —O—(CO)—NH—

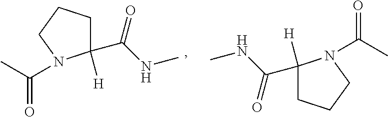

and/or —NH—(C═S)—NH groups (e.g.:

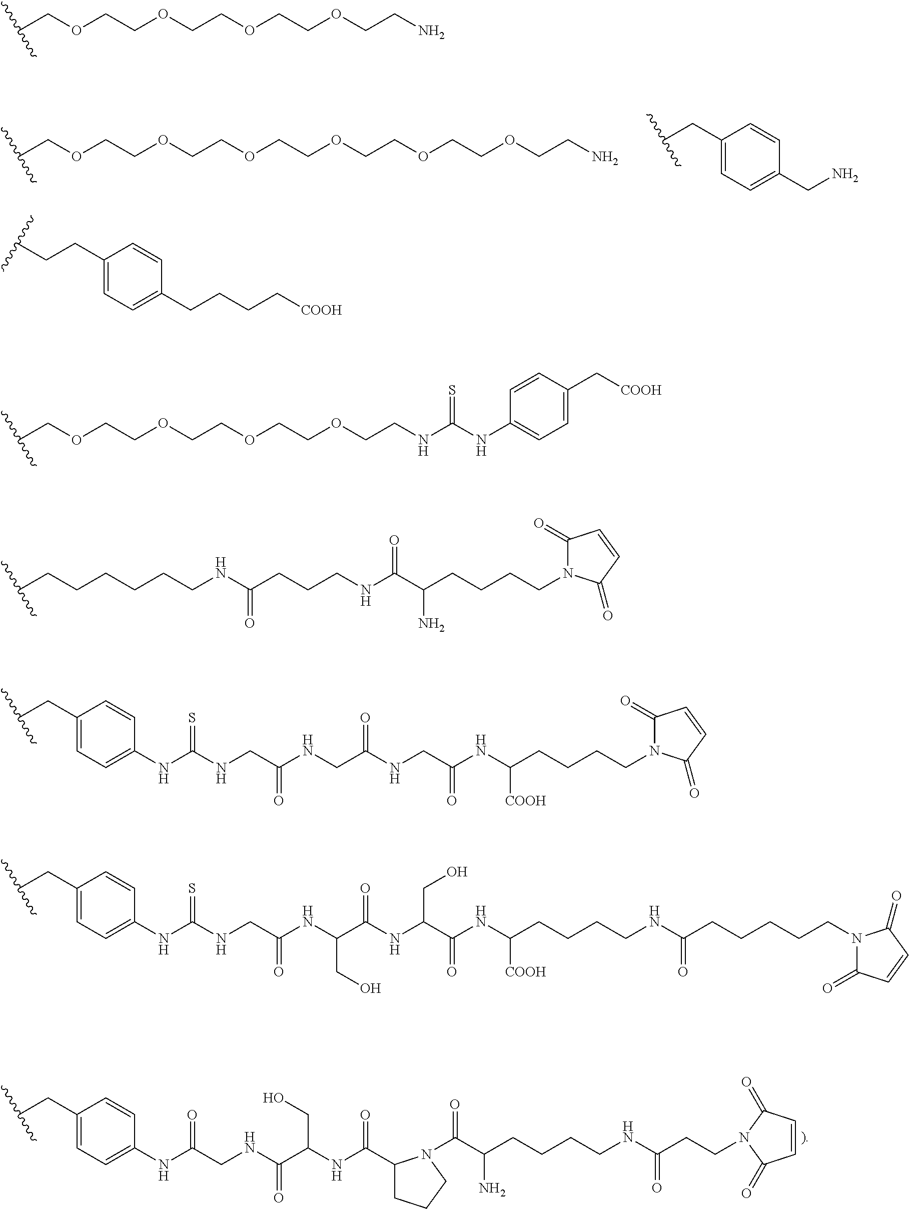

R can also stand for a functional group itself, such as, e.g., carboxyl, activated carboxyl, amino, nitro, isocyanate, isothiocyanate, hydrazine, semicarbazide, thiosemibarbazide, chloroacetamide, bromoacetamide, iodoacetamide, acryl, acylamino, mixed anhydrides, azide, acid chloride, acid bromide, hydroxide, sulfonyl chloride, vinyl sulfone, carbodiimide, maleimide or diazo (e.g.:

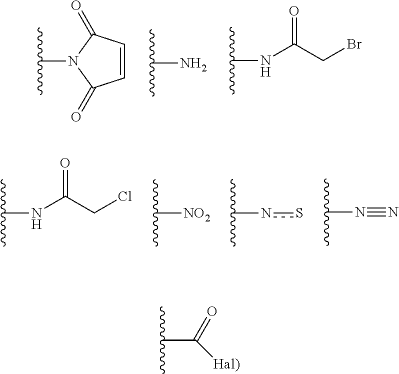

or another functional binding group.

A considerable number of the above-mentioned possible R-substituents allow a selective reaction with functional groups of the biomolecule in the optimal pH range, for example the addition to —SH groups (cysteine in the biomolecule), specifically only of —SH groups, e.g., on maleimides ((2,5-dioxo-2,5-dihydro-pyrrol-1-yl) compounds, see above) or bromoacetamides, if the coupling takes place in the weakly acidic pH range.

Activated carboxyl groups are defined as those carboxyl groups above that are derivatized such that they facilitate the reaction with a biomolecule. Which groups can be used for activation is known, and reference can be made, for example, to M. and A. Bodanszky, “The Practice of Peptide Synthesis,” Springerverlag 1984. Examples are aducts of carboxylic acid with carbodiimides or activated esters, such as, e.g., hydroxybenzotriazole esters. The activated carboxyl group for X is especially preferably selected from

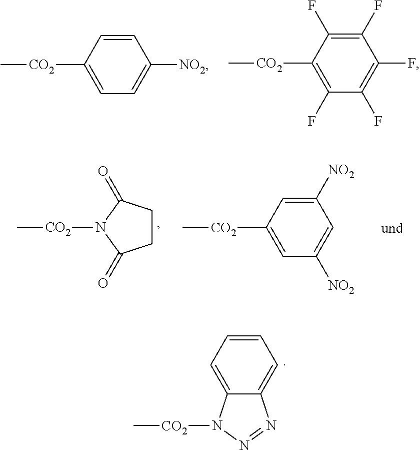

[and]

The activated esters of the above-described compounds are produced as known to one skilled in the art. For the case of isothiocyanates or α-haloacetates, the corresponding terminal amino precursors are reacted with thiophosgene or 2-halo-acetic acid halides according to methods that are known in the literature. Also, the reaction with correspondingly derivatized esters of N-hydroxysuccinimide, such as, for example:

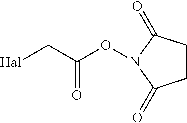

is possible (Hal=halogen).

In general, for this purpose, all commonly used activation methods for carboxylic acids can be used that are known in the prior art. If the R-substituent contains an amide group, the latter is produced, for example, by an activated carboxylic acid being reacted with an amine. The activation of the carboxylic acid is carried out according to commonly used methods. Examples of suitable activating reagents are dicyclohexylcarbodiimide (DCC), 1-ethyl-3-(3-dimethylaminopropyl)-carbodiimide-hydrochloride (EDC), benzotriazol-1-yloxytris(dimethylamino)-phosphoniumhexafluorophosphate (BOP) and O-(benzotriazol-1-yl)-1,1,3,3-tetramethyluroniumhexafluorophosphate (HBTU), preferably DCC. Also, the addition of O-nucleophilic catalysts, such as, e.g., N-hydroxysuccinimide (NHS) or N-hydroxybenzotriazole, is possible.

If the substituent is a carboxylic acid group, the latter can be used in protected form (e.g., in the form of benzyl ester), and the cleavage of the protective group can then be carried out hydrogenolytically.

To link this carboxylic acid group to a suitable functional group of a suitable biomolecule (for the description of biomolecules: see below), the latter should first be activated normally. Esters that are activated to this end are preferably produced at an intermediate stage, which then are attacked by a nucleophilic group of the biomolecule. In this way, a covalent linkage between the biomolecule and the compound of formula I is produced. Preferred activated esters are the esters of the N-hydroxysuccinimide, the esters of paranitrophenol or the esters of pentafluorophenol. If the functional group is to be linked in the form of an isothiocyanate to the biomolecule, first a terminal amine is preferably used, which, when necessary, can be provided with a suitable protective group. Suitable protective groups are known from the peptide chemistry. After cleavage of the protective group, the isothiocyanate can be produced by reaction of the primary terminal amine with thiophosgene. Nucleophilic groups of the biomolecule can be added to the latter.

The synthesis of the conjugates generally is carried out such that first a derivatized and functionalized ligand or chelate complex is produced, which then is linked to the biomolecule. It is also possible, however, that if synthetically produced biomolecules are used, the ligand or chelate complex according to the invention is incorporated in the biomolecules during the synthesis of the latter. This can be carried out, for example, during the sequential synthesis of oligopeptides on the synthesizing robots. If necessary, the protective groups that are commonly used in the synthesis of the corresponding biomolecule can be introduced into the compound according to the invention. The latter are then cleaved again in the synthesizer in line with the usual synthesis algorithm.

The compounds according to the invention contain at least two chirality centers (4- and 8-position). R can also contain one or more additional chirality centers, whereby in the descriptions and the claims, a distinction is not made between the various enantiomers, however, the above-mentioned compounds always comprise both enantiomers and in the presence of several stereocenters also all possible diastereomers as well as their mixtures.

“Biomolecule” is defined here as any molecule that either occurred naturally, for example in the body, or was produced synthetically with an analogous structure. Moreover, among the latter, those molecules are defined that can occur in interaction with a biological molecule that occurs, for example, in the body or a structure that occurs there, in such a way, for example, that the conjugates accumulate at specific desired spots of the body. “Body” is defined here as any plant or animal body, whereby animal and especially human bodies are preferred.

Biomolecules are especially the molecules that occur in living creatures that as products of an evolutionary selection by orderly and complex interactions meet specific objects of the organism and constitute the basis of its vital functions (changes in material and shape, reproduction, energy balance). In biomolecules, simple building blocks (amino acids, nucleobases, monosaccharides, fatty acids, etc.) of large molecules (proteins, nucleic acids, polysaccharides, lipids, etc.) are used in most cases. Corresponding macromolecules are also referred to as biopolymers.

The biomolecule advantageously can have, for example, a polypeptide skeleton that consists of amino acids with side chains that can participate in a reaction with the reactive group of the compounds according to the invention. Such side chains include, for example, the carboxyl groups of aspartic acid and glutamic acid radicals, the amino groups of lysine radicals, the aromatic groups of tyrosine and histidine radicals and the sulfhydryl groups of cysteine radicals.

A survey on biomolecules with numerous examples is found in the manuscript “Chemie der Biomoleküle [Chemistry of Biomolecules]” of TU-Graz (H. Berthold et al., Institut fur Organische Chemie [Institute for Organic Chemistry], TU-Graz, 2001), which can also be seen on the Internet under www.orgc.tu-graz.ac.at. The content of this document is integrated by reference in this description.

To form the conjugates according to the invention, the following biomolecules are especially suitable:

Biopolymers, proteins, such as proteins that have a biological function, HSA, BSA, etc., proteins and peptides, which accumulate at certain spots in the organism (e.g., in receptors, cell membranes, ducts, etc.), peptides that can be cleaved by proteases, peptides with predetermined synthetic sites of rupture (e.g., labile esters, amides, etc.), peptides that are cleaved by metalloproteases, peptides with photocleavable linkers, peptides with oxidative agents (oxydases) and cleavable groups, peptides with natural and unnatural amino acids, glycoproteins (glycopeptides), signal proteins, antiviral proteins and apoctosis, synthetically modified biopolymers, such as biopolymers that are derivatized with linkers, modified metalloproteases and derivatized oxydase, etc., carbohydrates (mono- to polysaccharides), such as derivatized sugars, sugars that can be cleaved in the organism, cyclodextrins and derivatives thereof, amino sugars, chitosan, polysulfates and acetylneuraminic acid derivatives, antibodies, such as monoclonal antibodies, antibody fragments, polyclonal antibodies, minibodies, single chains (also those that are linked by linkers to multiple fragments), red blood corpuscles and other blood components, cancer markers (e.g., CAA) and cell adhesion substances (e.g., Lewis X and anti-Lewis X derivatives), DNA and RNA fragments, such as derivatized DNAs and RNAs (e.g., those that were found by the SELEX process), synthetic RNA and DNA (also with unnatural bases), PNAs (Hoechst) and antisense, β-amino acids (Seebach), vector amines for transfer into the cell, biogenic amines, pharmaceutical agents, oncological preparations, synthetic polymers, which are directed to a biological target (e.g., receptor), steroids (natural and modified), prostaglandins, taxol and derivatives thereof, endothelins, alkaloids, folic acid and derivatives thereof, bioactive lipids, fats, fatty acid esters, synthetically modified mono-, di- and triglycerides, liposomes, which are derivatized on the surface, micelles that consist of natural fatty acids or perfluoroalkyl compounds, porphyrins, texaphrines, expanded porphyrins, cytochromes, inhibitors, neuramidases, neuropeptides, immunomodulators, such as FK 506, CAPE and gliotoxin, endoglycosidases, substrates that are activated by enzymes such as calmodulin kinase, casein-kinase II, glutathione-S-transferase, heparinase, matrix-metalloproteases, β-insulin-receptor-kinase, UDP-galactose 4-epimerase, fucosidases, G-proteins, galactosidases, glycosidases, glycosyltransferases and xylosidase, antibiotics, vitamins and vitamin analogs, hormones, DNA intercalators, nucleosides, nucleotides, lectins, vitamin B12, Lewis-X and related substances, psoralens, dienetriene antibiotics, carbacyclins, VEGF (vascular endothelial growth factor), somatostatin and derivatives thereof, biotin derivatives, antihormones, tumor-specific proteins and synthetic agents, polymers that accumulate in acidic or basic areas of the body (pH-controlled dispersion), myoglobins, apomyoglobins, etc., neurotransmitter peptides, tumor necrosis factors, peptides that accumulate in inflamed tissues, blood-pool reagents, anion- and cation-transporter proteins, polyesters (e.g., lactic acid), polyamides and polyphosphates.

Most of the above-mentioned biomolecules are commercially available from, for example, Merck, Aldrich, Sigma, Calibochem or Bachem.

In addition, all “plasma protein binding groups” or “target binding groups” that are disclosed in WO 96/23526 and WO 01/08712 can be used as biomolecules. The content of these two laid-open specifications is therefore integrated by reference into this description.

The number of the compounds of formula VIIa or VIIb according to the invention per biomolecule is in principle any number, but is preferably a molecular ratio of 0.3:1 to 11:1, in particular 0.5:1 to 7:1.

The compounds of formulas VIIa and b are also suitable for conjugation to all the molecules that are reacted with fluorescence dyes in the prior art to determine, for example, their location by epifluorescence microscopy within the cell. After the administration of the medication, the compounds with, in principle, any medications can also be conjugated to then track the transport within the organism by the NMR or scintigraphy technique. It is also possible that the conjugates from the compounds of formulas VIIa and b according to the invention and the biomolecules contain other additional molecules, which had been conjugated on the biomolecules. The term “biomolecule” in terms of this invention thus encompasses all molecules that occur in the biological systems and all molecules that are biocompatible (for the definition of biomolecules, see also above).

The relaxivity of the paramagnetic complexes according to the invention is so high that they are especially well suited for NMR diagnosis.

The compounds according to the invention bind to proteins. This property makes it possible for them, bonded to plasma proteins, to be retained longer in the blood stream and thus to make possible a visualization of the vascular space. Moreover, a visualization of sites of increased permeability, as they can be found in, for example, tumors, is also possible. This increased vascular permeability is in addition the basis of tumor therapy with radioactive metal complexes. The pharmaceutical agent leaves the vessel within the tumor, remains in the tissue and exposes the latter to its therapeutically effective radiation.

The plasma protein bond also makes possible an imaging diagnosis for locating infarctions or necroses because of the accumulation of substances according to the invention in the infarction or the necrosis.

Detecting, locating and monitoring necroses or infarctions is an important area in medicine. The myocardial infarction thus does not immediately result in an irreparable, non-functioning tissue, but rather introduces a dynamic process that stretches over an extended period (weeks to months). The disease proceeds in about three phases, which are not strictly separated from one another but rather are overlapping. The first phase, the development of the myocardial infarction, comprises the 24 hours after the infarction, in which the destruction such as a shock wave (wave front phenomenon) progresses from the subendocardium to the myocardium. The second phase, the already existing infarction, comprises the stabilization of the area, in which fiber formation (fibrosis) is carried out as a healing process. The third phase, the healed infarction, begins after all destroyed tissue is replaced by fibrous scar tissue. During this period, an extensive restructuring takes place.

For the evaluation of a myocardial infarction, it is of decisive importance to know how large the proportion of the tissue that is definitively lost in the infarction is and at what point the loss occurred, since the type of therapy depends on this knowledge.

Infarctions are carried out not only in the myocardium, but also in other tissues, such as in the brain or in the kidney.

While the infarction can be healed to a certain extent, only the harmful sequelae for the residual organism can be prevented or at least mitigated in the case of a necrosis, locally limited tissue death. Necroses can develop in many ways: by injuries, chemicals, oxygen deficiency or by radiation. As in infarction, the knowledge of the extent and type of a necrosis is important for additional medical treatment.

The production of the compounds of general formulas VIIa and b according to the invention is carried out in that in a way that is known in the art, the compounds of general formulas VII'a and VII'b

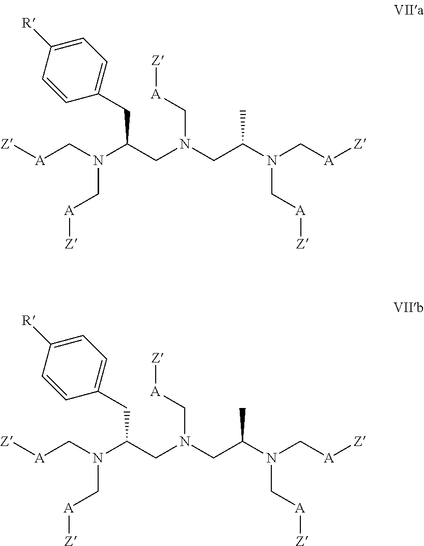

in which Z′ means a carboxyl protective group, protective groups Z′ are cleaved, and the thus obtained acids are reacted in a way that is known in the art with at least one metal oxide or metal salt of an element of atomic numbers 21-29, 31, 32, 37-39, 42-44, 46, 47, 49, 58-71, 75, 77, 82 or 83, and then, if desired, acid hydrogen atoms that are present with inorganic and/or organic acid or amino acids are converted into physiologically compatible salts.

As carboxyl protective groups Z′, lower alkyl, aryl and aralkyl groups are suitable, for example, the methyl, ethyl, propyl, butyl, phenyl, benzyl, diphenylmethyl, triphenylmethyl, or bis(4-nitrophenyl)-methyl group as well as trialkylsilyl groups.

The cleavage of protective groups Z′ is carried out in a way that is known in the art, for example by hydrolysis, alkaline saponification of esters, preferably with alkali in aqueous-alcoholic solution at temperatures of 0° C. to 50° C. or in the case of benzyl esters by catalytic hydrogenation and in the case of t-butyl esters by acidic hydrolysis, for example with hydrochloric acid or trifluoroacetic acid (Protective Groups in Organic Synthesis, 2^{nd} Edition, T. W. Greene and P. G. M. Wutz, John Wiley & Sons, Inc., New York, 1991).

The production of the compounds of formulas VIIa and VIIb according to the invention is explained below in the example of the selected compound 1, in which Z′ is t-butyl.

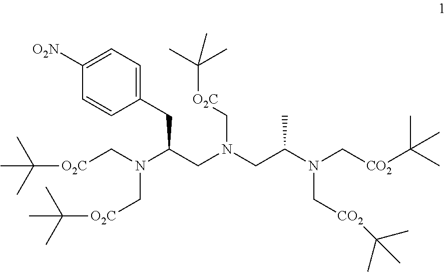

By alkaline saponification and subsequent ion-exchange treatment, compound 1 can be converted into compound VII'a with free carboxylic acids.

Compound 1 is produced from triamine 2 by an alkylation reaction with bromoacetic acid-tert-butyl ester in an acetonitrile/water mixture with potassium carbonate as a base.

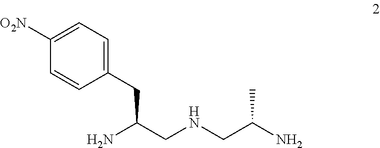

Compound 2 is accessible by reduction of amide 3 with a borane-tetrahydrofuran complex. In this case, the conditions as they are described in, for example, J. Amer.

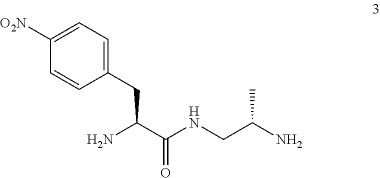

Chem. Soc., (1990), 9608, are followed.

Amide 3 is produced from the 2× Boc-protected diamine 4 by reaction with trifluoroacetic acid and dichloromethane.

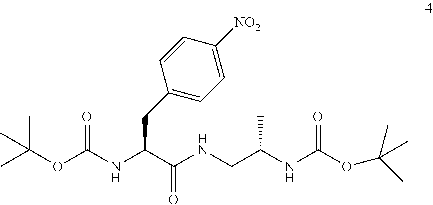

The formation of amide 4 is carried out in this case according to the methods that are well-known to one skilled in the art, for example, acid activation by
Oxalyl chloride: J. Org. Chem., 29: 843 (1964)
Thionyl chloride: Helv., 42: 1653 (1959)
Carbodiimide. Helv. 46: 1550 (1963)
Carbodiimide/Hydroxysuccinimide: J. Am. Chem. Soc. 86: 1839 (1964) as well as J. Org. Chem. 53: 3583 (1988); Synthesis 453 (1972)
Anhydride Method: 2-Ethoxy-l-ethoxycarbonyl-1,2-dihydroquinoline: J. Am. Chem. Soc. 90:1651 (1986); Int. J. Pept. Prot. Res., 26:493 (1985); Am. Soc. 73: 3547 (1951)
Imidazolide Method: Am. Soc. 91:2691 (1969)
J. Med. Chem. 1996, 392596; Tetrahedron Letters 1994, 35, 5981; Bioorg. Med. Letters 1996, 6, 55; J. Chem. Soc. Commun. 1994, 201,
from the commercially available acid 5 and the mono-protected amine 6.

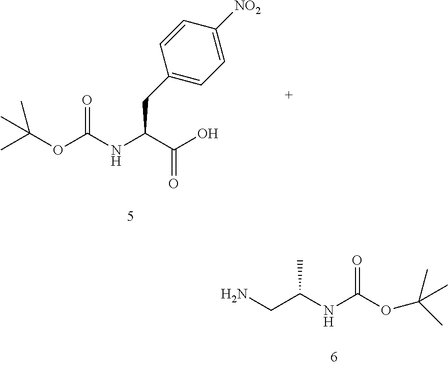

Compound 6 is produced by reduction of azide 7 with hydrogen and Pd/C in ethyl acetate.

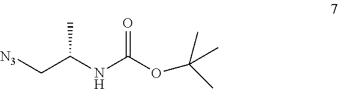

Compound 7 is described in the substitution reaction of mesylate 8 with sodium azide.

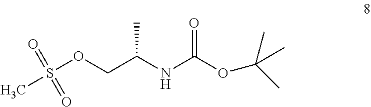

Mesylate 8 can be obtained by reaction with alcohol 9 and methanesulfonic acid

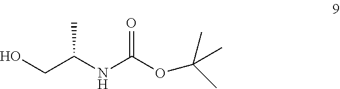

chloride.

Alcohol 9 is the product from a reaction of commercially available (S)-2-amino-1-propanol (10) and di-tert-butyldicarbonate [(Boc)₂O] in tetrahydrofuran.

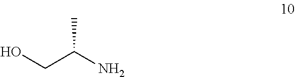

The nitro group that is contained in compound 1 is used after their conversion into the amino group directly as a binding site for biomolecules, for example via an amide formation with the aid of activated esters or reductive amination with carbonyl groups or after conversion into selectively reacting groups. These conversion reactions are well-known to one skilled in the art.

Gansow (EP 484984) and Meares (U.S. Pat. No. 4,622,420) thus describe the visualization of haloacetamides of acyclic complexing agents, which are used for coupling with —SH groups or —NH₂ groups.

The isothiocyanato group makes possible a selective coupling with amino groups. Its visualization and the reaction with amines to form the corresponding thioureas has been described in, for example, U.S. Pat. No. 4680338 of Immunomedics. The reaction with hydrazides to form thio-semicarbazides is described in Application WO 95/15335 of the Neorx Corp.

Maleimides make possible a selective reaction with —SH groups. Their visualization and reaction are described in, for example, the patents U.S. Pat. No. 5,273,743 and EP 446071 by Hybritech or in EP 345723 of Nihon-Medi Physics.

The introduction of the desired metal ions of complexes for the production of NMR diagnostic agents can be carried out in the way as it was disclosed in Patents EP 71564, EP 130934 and DE-OS 34 01 052. To this end, the metal oxide or a metal salt (for example a chloride, nitrate, acetate, carbonate or sulfate) of the desired element is dissolved or suspended in water and/or a lower alcohol (such as methanol, ethanol, or isopropanol) and reacted with the solution or suspension of the equivalent amount of the complexing agent according to the invention.

The neutralization of optionally still present free carboxy groups is carried out with the aid of inorganic bases (e.g., hydroxides, carbonates or bicarbonates) of, e.g., sodium, potassium, lithium, magnesium or calcium and/or organic bases, such as, i.a., primary, secondary and tertiary amines, such as, e.g., ethanolamine, morpholine, glucamine, N-methylglucamine and N,N-dimethylglucamine, as well as basic amino acids, such as, e.g., lysine, arginine and ornithine or amides of originally neutral or acidic amino acids.

For the production of neutral complex compounds, as much of the desired base can be added, for example, into acid complex salts in aqueous solution or suspension so that the neutral point is reached. The solution that is obtained can then be evaporated to the dry state in a vacuum. It is often advantageous to precipitate the neutral salts that are formed by adding water-miscible solvents, such as, e.g., lower alcohols (methanol, ethanol, isopropanol, etc.), lower ketones (acetone, etc.), polar ethers (tetrahydrofuran, dioxane, 1,2-dimethoxyethane, etc.) and thus to obtain easily isolated and readily purified crystallizates. It has proven especially advantageous to add the desired base as early as during the complexing of the reaction mixture and thus to save a process step.

If the complexing agents are used for the production of radiodiagnostic agents or therapeutic agents, the production of the complexes from the complexing agents can be carried out according to the methods that are described in “Radiotracers for Medical Applications,” Vol I, CRC Press, Boca Raton, Fla. (1983).

It may be desirable to produce the complex only shortly before its use, especially if it is to be used as a radiopharmaceutical agent. The invention therefore also comprises a kit for the production of radiopharmaceutical agents, comprising a compound of formula VIIa or VIIb, in which Z stands for a radioisotope.

Subjects of the invention are also pharmaceutical agents that contain at least one physiologically compatible compound of general formula VIIa or VIIb, optionally with the additives that are commonly used in galenicals.

The production of the pharmaceutical agents according to the invention is carried out in a way that is known in the art by the complex compounds according to the invention—optionally with the addition of the additives that are commonly used in galenicals—being suspended or dissolved in aqueous medium, and then the suspension or solution optionally being sterilized. Suitable additives are, for example, physiologically harmless buffers (such as, e.g., tromethamine), additives of complexing agents or weak complexes (such as, e.g., diethylenetriaminepentaacetic acid or the Ca complexes that correspond to the metal complexes according to the invention) or—if necessary—electrolytes such as, e.g., sodium chloride or—if necessary—antioxidants such as, e.g., ascorbic acid.

If suspensions or solutions of the agents according to the invention in water or physiological salt solution are desired for enteral administration or other purposes, they are mixed with one or more adjuvant(s) that are commonly used in galenicals [e.g., methyl cellulose, lactose, mannitol] and/or surfactant(s) [e.g., lecithins, Tween®, Myrj®] and/or flavoring substance(s) for taste correction [e.g., ethereal oils].

In principle, it is also possible to produce the pharmaceutical agents according to the invention even without isolating the complex salts. In each case, special care must be taken to perform the chelation such that the salts and salt solutions according to the invention are virtually free of non-complexed metal ions that have a toxic effect.

This can be ensured, for example, with the aid of color indicators, such as xylenol orange, by control titrations during the production process.

The invention therefore also relates to a process for the production of complex compounds and their salts. As a final precaution, there remains purification of the isolated complex salt.

The pharmaceutical agents according to the invention preferably contain 1 fmol-1.3 mol/l of the complex salt and are generally dosed in amounts of 0.5 pmol/kg-5 mmol/kg. They are intended for enteral and parenteral administration. The complex compounds according to the invention are used
1. For NMR diagnosis in the form of their complexes with the paramagnetic ions of the elements with atomic numbers 21-29, 42, 44 and 58-70. Suitable ions are, for example, the chromium(III), ion(II), cobalt(II), nickel(II), copper(II), praseodymium(III), neodymium(III), samarium(III) and ytterbium(III) ions. Because of their strong magnetic moment, the gadolinum(II), terbium(III), dysprosium(III), holmium(III), erbium(III), manganese (II) and iron(III) ions are especially preferred for NMR diagnosis.
2. For radiodiagnosis and radiotherapy in the form of their complexes with the radioisotopes of the elements with atomic numbers 26, 27, 29, 31, 32, 37-39, 43, 46, 47, 49, 61, 62, 64, 67, 70, 71, 75, 77, 82 and 83.

The agents according to the invention meet the many different requirements for suitability as contrast media for nuclear spin tomography. After oral or parenteral administration, they are thus extremely well suited for enhancing the informational value of the image that is obtained with the aid of a nuclear spin tomograph by increasing the signal intensity. They also show the high effectiveness that is necessary to load the body with the smallest possible amounts of foreign substances and the good compatibility that is necessary to maintain the non-invasive nature of the studies.

The good water solubility and low osmolality of the agents according to the invention allow for the production of highly concentrated solutions so as to keep the volume burden of the circulatory system within reasonable limits and to offset the dilution by bodily fluids, i.e., NMR diagnostic agents have to be 100 to 1000 times more water-soluble than for NMR spectroscopy. In addition, the agents according to the invention have not only a high stability in vitro but also a surprisingly high stability in vivo, so that a release or an exchange of the ions, which are inherently toxic and not covalently bonded in the complexes, is carried out only extremely slowly within the time that it takes for the new contrast media to be completely excreted again.

In general, the agents according to the invention are dosed for use as NMR diagnostic agents in amounts of 0.0001-5 mmol/kg, preferably 0.005-0.5 mmol/kg. Details of use are discussed in, e.g., H.-J. Weinmann et al., Am. J. of Roentgenology 142, 619 (1984).

Low dosages (below 1 mg/kg of body weight) of organ-specific NMR diagnostic agents can be used, for example, for detecting tumors and myocardial infarctions. Especially low dosages of the complexes according to the invention are suitable for use in radiotherapy and radiodiagnosis. Thus, both for therapeutic and diagnostic purposes, dosages of 0.5 pM/kg-5 μmol/kg, preferably 50 pM/kg-500 nmol/kg are used. With respect to the radioactive metal ion, about 100-100,000× smaller molar concentrations are usually used than that which is the case for the chelating agents or chelating agent-bioconjugates, such that the chelating agents or chelating agent-bioconjugates are present in excess.

The complex compounds according to the invention can also advantageously be used as susceptibility reagents and as shift reagents for in-vivo NMR spectroscopy.

The agents according to the invention are also suitable as radiodiagnostic agents and radiotherapeutic agents based on their advantageous radioactive properties and the good stability of the complex compounds that are contained therein. Details of their use and dosage are described in, e.g., “Radiotracers for Medical Applications,” CRC Press, Boca Raton, Fla. 1983, as well as in Eur. J. Nucl. Med. 17 (1990) 346-364 and Chem. Rev. 93 (1993) 1137-1156.

For SPECT, the complexes with isotopes ¹¹¹In and ⁹⁹ⁿTc are suitable.

Another imaging method with radioisotopes is the positron-emission tomography, which uses positron-emitting isotopes such as, e.g., ⁴³Sc, ⁴⁴Sc, ⁵²Fe, ⁵⁵Co, ⁶⁸Ga, ⁶⁴Cu, ⁸⁶Y and ^{94m}Tc (Heiss, W. D.; Phelps, M. E.; Positron Emission Tomography of Brain, Springer Verlag Berlin, Heidelberg, N.Y. 1983).

The compounds according to the invention are also suitable, surprisingly enough, for differentiating malignant and benign tumors in areas without blood-brain barriers.

They are also distinguished in that they are completely eliminated from the body and thus are well-tolerated.

Since the substances according to the invention accumulate in malignant tumors (no diffusion in healthy tissue, but high permeability of tumor vessels), they can also support the radiation therapy of malignant tumors. The latter is distinguished from the corresponding diagnosis only by the amount and type of the isotope that is used. The purpose in this case is the destruction of tumor cells by high-energy short-wave radiation with the lowest possible range of action. For this purpose, interactions of the metals that are contained in the complexes (such as, e.g., iron or gadolinium) with ionizing radiations (e.g., x rays) or with neutron rays are employed. By this effect, the local radiation dose at the site where the metal complex is found (e.g., in tumors) increases significantly. To produce the same radiation dose in the malignant tissue, radiation exposure for healthy tissue can be considerably reduced and thus burdensome side effects for the patients can be avoided when such metal complexes are used. The metal complex conjugates according to the invention are therefore also suitable as radio-sensitizing substances in the radiation therapy of malignant tumors (e.g., exploiting Mössbauer effects or in the case of neutron capture therapy). Suitable β-emitting ions are, e.g., ⁴⁶Sc, ⁴⁷Sc, ⁴⁸Sc, ⁷²Ga, ⁷³Ga, ⁹⁰Y, ⁶⁷Cu, ¹⁰⁹Pd, ¹¹¹Ag, ¹⁴⁹Pm, ¹⁵³Sm, ¹⁶⁶Ho, ¹⁷⁷Lu, ¹⁸⁶Re, and ¹⁸⁸Re, whereby ⁹⁰Y, ¹⁷⁷Lu, ⁷²Ga, ¹⁵³Sm and ⁶⁷Cu are preferred. Suitable α-emitting ions that have short half-lives are, e.g., ²¹¹At, ²¹¹Bi, ²¹²Bi, ²¹³Bi and ²¹⁴Bi, whereby ²¹²Bi is preferred. A suitable photon- and electron-emitting ion is ¹⁵⁸Gd, which can be obtained from ¹⁵⁷Gd by neutron capture.

If the agent according to the invention is intended for use in the variant of the radiation therapy that is proposed by R. L. Mills et al. [Nature Vol. 336 (1988), p. 787], the central ion must be derived from a Mössbauer isotope, such as, for example, ⁵⁷Fe or ¹⁵¹Eu.

In the in-vivo administration of the therapeutic agents according to the invention, the latter can be administered together with a suitable vehicle, such as, e.g., serum, or physiological common salt solution, and together with another protein, such as, e.g., human serum albumin. In this case, the dosage depends on the type of cellular destruction, the metal ion that is used and the type of imaging method.

The therapeutic agents according to the invention are administered preferably parenterally, preferably i.v.

Details of use of radiotherapeutic agents are discussed in, e.g., R. W. Kozak et al. TIBTEC, October 1986, 262 (see also Bioconjugate Chem. 12 (2001) 7-34).

Viewed overall, it has also been possible to synthesize new complexing agents, metal complexes and metal complex salts which open up improved possibilities in diagnostic and therapeutic medicine.

The examples below are used for a more detailed explanation of the subject of the invention.

Examples 10, 12-15, 18, 19, 20, 21, and 28-31 describe conjugates with antibodies. Conjugates with other biomolecules can be produced according to the following general operating instructions:

Here, “AAV” stands for general operating instructions, “RP-18” refers to a “reversed-phase” stationary chromatography phase. The number of complexes per biomolecule was determined by means of scintigraphy or ICP (inductively coupled plasma atomic emission spectroscopy).

### General Operating Instructions (AAV) I: Albumin-Amide Conjugates

3 mmol of the acid is dissolved in 15 ml of DMF, mixed with 380 mg (3.3 mmol) of N-hydroxysuccinimide and 681 mg of dicyclohexylcarbodiimide while being cooled with ice and preactivated for 1 hour in ice. The active ester mixture is added in drops within 30 minutes in a solution of 16.75 g (0.25 mmol) of bovine serum albumin (BSA) in 150 ml of phosphate buffer (pH 7.4) and stirred for 2 hours at room temperature. The batch solution is filtered, the filtrate is ultrafiltered with an AMICON® YM30 (cut-off 30,000 Da), the retentate is chromatographed on a Sephadex® G50 column, and the product fractions are freeze-dried.

### General Operating Instructions (AAV) II: Albumin-Maleimide Conjugates

0.0438 mmol of the maleimide in 1 ml of DMF is added to 0.84 g (0.0125 mmol) of bovine serum albumin (BSA), dissolved in 15 ml of phosphate buffer (pH 7.4), and it is stirred for one hour at room temperature. The batch solution is filtered, the filtrate is ultrafiltered with an AMICON® YM30 (cut-off 30,000 Da), the retentate is chromatographed on a Sephadex® G50 column, and the product fractions are freeze-dried.

### General Operating Instructions (AAV) III: Production of Amide Conjugates

3 mmol of the acid is dissolved in 15 ml of DMF, mixed with 380 mg (3.3 mmol) of N-hydroxysuccinimide and 681 mg of dicyclohexylcarbodiimide while being cooled with ice, and preactivated for 1 hour in ice. The active ester mixture is added in drops to a solution of 2.5 mmol of amine components in 15-150 ml of DMF and stirred overnight at room temperature. The batch solution is filtered and chromatographed on silica gel.

### General Operating Instructions (AAV) IV: Production of Maleimido-SH Conjugates

3 mmol of the maleimide in 15 ml of DMF is added in drops to 2.5 mmol of SH components in 15-150 ml of DMF, and it is stirred for one hour at room temperature. The batch solution is filtered, the filtrate is ultrafiltered with an AMICON® YM30 (cut-off 30,000 Da), the retentate is chromatographed on a Sephadex® G50 column, and the product fractions are freeze-dried.

### General Operating Instructions (AAV) V: Production of Haloacetamido-SH Conjugates

3 mmol of the haloacetamide in 15 ml of DMF is added in drops to 2.5 mmol of SH components in 15-150 ml of DMF, and it is stirred for eight hours at room temperature. The batch solution is filtered, the filtrate is ultrafiltered with an AMICON® YM30 (cut-off 30,000 Da), the retentate is chromatographed on a Sephadex® G50 column, and the product fractions are freeze-dried.

### EXAMPLE 1

### a) (S)-(2-Hydroxy-1-methyl-ethyl)-carbamic acid tert-butyl ester

10.50 g (140 mmol) of (S)-(+)-2-amino-1-propanol was dissolved in 110 ml of THF and cooled at 0° C. A solution of 30.2 (139 mmol) of di-tert.-butyldicarbonate in 45 ml of THF was added in drops to this stirred solution. The reaction mixture was stirred for 90 minutes at 25° C. and concentrated by evaporation in a rotary evaporator. The residue was taken up in 300 ml of diethyl ether and then washed with 90 ml of 0.01 M HCl solution. The organic phase was dried by means of sodium sulfate and concentrated by evaporation in a rotary evaporator and at the oil pump. The crude product (20.4 g) was used without further purification for the following reaction.

### b) (S)-Methanesulfonic acid 2-tert-butoxycarbonylamino-propyl ester

20.3 g (116 mmol) of la was dissolved in 125 ml of dichloromethane and mixed with 17.7 g (175 mmol) of triethylamine. A solution of 14.7 g (128 mmol) of methanesulfonic acid chloride in 30 ml of dichloromethane was added in drops at 0° C. The reaction solution was stirred at 0° C. for 2 hours and then mixed with 300 ml of water. The organic phase was separated. The aqueous phase was extracted twice with 150 ml of dichloromethane. The combined organic phases were washed once with 150 ml of 0.1 M HCl solution, twice with 150 ml each of 5% sodium bicarbonate solution and finally with 50 ml of aqueous, saturated sodium chloride solution. The organic phase was dried with sodium sulfate. The solution was concentrated by evaporation and crystallized out in a refrigerator. The crystals were washed with cold hexane. The desired product was produced with a yield of 25.4 g (100 mmol).

MS-FAB: 254 (M⁺+1.13)

### c) (S)-(2-Azido-1-methyl-ethyl)-carbamic acid tert-butyl ester

A solution of 20.3 g (100 mmol) of 1b in 155 ml of DMSO was mixed with 7.8 g (120 mmol) of sodium azide and stirred for 24 hours at 45° C. 250 ml of ice water was added. The mixture was extracted several times with 200 ml of dichloromethane. The combined organic phases were washed twice with 50 ml of aqueous, saturated, sodium chloride solution. The organic phase was concentrated by evaporation. 11.4 g of crude product was produced. The crude product was used without further purification for the following reaction.

MS-FAB: 201 (M⁺+1.23)

### d) (S)-(2-Amino-1-methyl-ethyl)-carbamic acid tert-butyl ester

A stirred solution of 11.4 g (57 mmol) of 1c in 165 ml of ethyl acetate was mixed with 1.8 g of Pd/C (10%) and exposed for 15 hours to a hydrogen atmosphere of 4 bar. The catalyst was separated by filtration (so-called G4 frit). The filtrate was concentrated by evaporation in a rotary evaporator and purified by column chromatography (SiO₂-dichloromethane→dichloromethane:methanol 1:1). The desired product was produced with a yield of 73% (7.25 g; 42 mmol).

MS-FAB: 175 (M⁺+1.29)

### e) (S,S)-{2-[2-tert-Butoxycarbonylamino-3-(4-nitro-phenyl)-propionylamino]-1-methyl-ethyl}-carbamic acid tert-butyl ester

A stirred solution of 7.2 g (41 mmol) of 1d, 245 ml of water and 245 ml of dichloromethane was mixed with commercially available 12.9 g (42 mmol) of (S)-2-tert-butoxycarbonylamino-3-(4-nitro-phenyl)-propionic acid (Bachem) and 6.4 g (42 mmol) of 1-hydroxybenzotriazole—H₂O (HOBT). The solution was cooled to 0° C. and mixed with 8.8 g (46 mmol) of 1-(dimethylaminopropyl)-3-ethylcarbodiimide (EDCI). The reaction solution was stirred for seven hours at 0° C., for 12 hours at room temperature, and for 24 hours at 60° C. The solution was cooled to room temperature. The aqueous phase was separated and extracted several times with dichloromethane. The combined organic phases were washed with 5% sodium bicarbonate solution and aqueous, saturated sodium chloride solution. It was dried with sodium sulfate and concentrated by evaporation in a rotary evaporator. The residue was mixed with hexane, torn apart, suctioned off and rewashed with cold hexane. The solid was dried in a vacuum at 30° C. The desired product 1e was produced with a yield of 77% (14.9 g; 32 mmol).

MS-FAB: 467 (M⁺+1.38)

### f) (S,S)-N-(2-Amino-propyl)-3-(4-nitro-phenyl)-propane-1,2-diamine

14.9 g (32 mmol) of 1 e was suspended in 180 ml of dichloromethane. Then, 54.2 g (475 mmol) of trifluoroacetic acid was added in drops. The solution was stirred for one hour and concentrated by evaporation in a rotary evaporator. Dichloromethane was added, and it was concentrated by evaporation once more. Diethyl ether was added. The precipitating solid was separated and washed with cold diethyl ether. The solid was dried in a vacuum at 35° C. 225 ml of (1 M) borane-tetrahydrofuran complex was added in drops to a stirred solution of this solid (17.7 g) in 225 ml of abs. THF. The reaction solution was refluxed for 6 hours and then stirred overnight at room temperature. 60 ml of methanol was carefully added in drops, and it was stirred for another 2 hours. The solution was concentrated by evaporation in a rotary evaporator and mixed with 150 ml of ethanol. HCl gas was introduced, so that a solid precipitated. It was concentrated by evaporation and mixed with dry diethyl ether. The solid was separated, washed with cold diethyl ether and dried in a vacuum at 40° C. 9.61 g (˜26.6 mmol) of the desired product 1f was produced as trihydrochloride.

MS-FAB: 253 (M⁺+1.28)

### g) (S,S)-{[2-{[2-(Bis-tert-butoxycarbonylmethyl-amino)-propyl]tert-butoxycarbonylmethyl-amino}-1-(4-nitro-benzyl)-ethyl]-tert-butoxycarbonylmethyl-amino-acetic acid tert-butyl ester

43.8 g (317 mmol) of potassium carbonate and 39 g (200 mmol) of bromoacetic acid-tert-butyl ester were added to a stirred solution of 9.6 g (27 mmol) of if in 290 ml of acetonitrile and 60 ml of water. The solution was heated for ˜7 hours to 70° C. Another 13.1 g (94 mmol) of potassium carbonate and 8.8 ml (60 mmol) of bromoacetic acid-tert-butyl ester were added. The solution was stirred for ˜7 hours at 70° C. 4.3 g (26 mmol) of potassium iodide was added. The solution was stirred for ˜7 hours at 70° C. The reaction solution was concentrated by evaporation in a rotary evaporator, mixed with water and extracted three times with ethyl acetate. The combined organic phases were washed with aqueous, saturated sodium chloride solution, dried with sodium sulfate and concentrated by evaporation. The residue was purified by column chromatography (SiO₂-dichloromethane→dichloromethane:methanol 98:2). The desired product, 1 g, was produced in a yield of 54% (23.2 g; 42 mmol).

MS-FAB: 824 (M⁺+1.58)

### h) (S,S))-{[2-{[2-(Bis-carboxymethyl-amino)-propyl]-carboxymethyl-amino}-1-(4-nitro-benzyl)-ethyl]-carboxymethyl-amino}-acetic acid

1.65 g (2 mmol) of 1g was added to a stirred solution of 14.8 g (130 mmol) of trifluoroacetic acid, 25.5 g (300 mmol) of dichloromethane and 2.9 g (25 mmol) of triethylsilane. The reaction mixture was stirred for 1 hour and concentrated by evaporation in a rotary evaporator and at the oil pump. The residue was mixed with diethyl ether. The solid was separated by filtration and washed with diethyl ether. The desired product 1h was produced with a yield of 99% (1.0 g; 1.99 mmol).

MS-FAB: 543 (M⁺+1.59)

### EXAMPLE 2

### (S,S))-{[2-(4-Amino-phenyl)-1-({[2-(bis-tert-butoxycarbonylmethyl-amino)-propyl]- tert-butoxycarbonylmethyl-amino}-methyl)-ethyl]-tert-butoxycarbonylmethyl-amino}-acetic acid tert-butyl ester

0.2 g of Pd/C (10%) was added to a solution of 0.5 g (0.6 mmol) of 1g in 10 ml of isopropanol. The atmosphere over the reaction solution was provided with hydrogen. The solution was stirred for 5 hours, filtered and concentrated by evaporation. The desired product 2 was produced in 81% (397 mg; 0.5 mmol).

MS-FAB: 795 (M⁺+1.63)

### EXAMPLE 3

### (S,S)-[(1-(4-Amino-benzyl)-2-{[2-(bis-carboxymethyl-amino)-propyl]-carboxymethyl-amino}-ethyl)-carboxymethyl-amino]-acetic acid

Method A: 0.2 g of Pd/C (10%) was added to a solution of 0.5 g (0.9 mmol) of lh in 10 ml of isopropanol. The atmosphere over the reaction solution was provided with hydrogen. The solution was stirred for 5 hours, filtered and concentrated by evaporation. The desired product 3 was produced in a yield of 87% (410 mg; 0.78 mmol).

MS-FAB: 513 (M⁺+1.43)

Method B: 397 mg (0.5 mmol) of 2 was added to a stirred solution of 3.64 g (32 mmol) of trifluoroacetic acid, 6.38 g (75 mmol) of dichloromethane and 0.7 g (6 mmol) of triethylsilane. The reaction mixture was stirred for 1 hour and concentrated by evaporation in a rotary evaporator and at the oil pump. The residue was mixed with diethyl ether. The solid was separated by filtration and washed with diethyl ether. The desired product 3 was produced with a yield of 99% (253 mg; 0.5 mmol).

MS-FAB: 513 (M⁺+1.48)

### EXAMPLE 4

### (S,S)-[(2-{[2-(Bis-carboxymethyl-amino)-propyl]-carboxymethyl-amino}-1-{4-[3-(2,5-dioxo-2,5-dihydro-pyrrol-1-yl)-propionylamino]-benzyl}-ethyl)-carboxymethyl-amino]-acetic acid

800 mg (3 mmol) of 3-(2,5-dioxo-2,5-dihydro-pyrrol-1-yl)-propionic acid 2,5-dioxo-pyrrolidin-1-yl ester (Aldrich) at 0° C. was added to a stirred solution of 1.02 g (2 mmol) of 3 and 774 mg (6 mmol) of diisopropylethylamine in 20 ml DMF. The reaction solution was stirred for 4 hours at room temperature. The solution was added drop by drop to 120 ml of vigorously stirred diethyl ether. The suspension was stirred for 30 minutes and filtered. The residue was dried at the oil pump. A portion of the residue was purified by semi-preparative RP-HPLC.

MS-FAB: 664 (M⁺+1.24)

### EXAMPLE 5

### (S,S)-[(1-(4-Isothiocyanato-benzyl)-2-{[2-(bis-carboxymethyl-amino)-propyl]-carboxymethyl-amino}-ethyl)-carboxymethyl-amino]-acetic acid

114 mg (1 mmol) of thiophosgene in a little chloroform is added in drops at 0° C. to a vigorously-stirred two-phase system that consists of 0.51 g (1 mmol) of 3, 3 ml of water, 605 mg (6 mmol) of triethylamine and 3 ml of chloroform. The solution was stirred for 3 hours. The organic phase was separated. The organic phase was extracted twice with water. The combined, aqueous phases were washed with dichloromethane, diluted with water and freeze-dried. The substance was checked for purity by HPLC: HyPurity C18 (5 □m, 150×3.0 mm) with acetonitrile-:water-:trifluoroacetic acid gradient (3:96.9:0.1→99.9:0:0.1). The desired product 5 was produced with a yield of 91% (505 mg, 910 mmol).

MS-FAB: 555 (M⁺+1.39)

### EXAMPLE 6

### (S,S)-({2-{[2-(Bis-carboxymethyl-amino)-propyl]-carboxymethyl-amino}-1-[4-(2-bromo-acetylamino)-benzyl]-ethyl}-carboxymethyl-amino)-acetic acid

202 mg (1.1 mmol) of bromoacetic acid bromide was added at −20° C. to a solution of 0.51 g (1 mmol) of 3 and 606 mg (6 mmol) of triethylamine in 10 ml DMF. The reaction solution was stirred for one hour at room temperature. The solution was poured into vigorously-stirred diethyl ether. The precipitate was filtered off, taken up in water and immediately freeze-dried. The substance was checked for purity by HPLC: HyPurity C18 (5 μm, 150×3.0 mm) with acetonitrile-:water-:trifluoroacetic acid gradient (3:96.9:0.1→99.9:0:0.1). The desired product 6 was produced with a yield of 82% (520 mg, 820 μmol).

MS-FAB: 634 (M⁺+1.46)

### EXAMPLE 7

### (S,S)-({2-{[2-(Bis-carboxymethyl-amino)-propyl]-carboxymethyl-amino}-1-[4-(2-iodo-acetylamino)-benzyl]-ethyl}-carboxymethyl-amino)-acetic acid

274 mg (1.1 mmol) of iodoacetic acid bromide was added at −20° C. to a solution of 0.51 g (1 mmol) of 3 and 606 mg (6 mmol) of triethylamine in 10 ml of DMF. The reaction solution was stirred for one hour at room temperature. The solution was poured into vigorously-stirred diethyl ether. The precipitate was filtered off, taken up in water and immediately freeze-dried. The substance was checked for purity by HPLC: HyPurity C18 (5 μm, 150×3.0 mm) with acetonitrile-:water-:trifluoroacetic acid gradient (3:96.9:0.1→99.9:0:0.1). The desired product 7 was produced with a yield of 88% (600 mg, 880 μmol).

MS-FAB: 681 (M⁺+1.32)

### EXAMPLE 8

### (S,S)-({2-{[2-(Bis-carboxymethyl-amino)-propyl]-carboxymethyl-amino}-1-[4-(2-iodo-acetylamino)-benzyl]-ethyl}-carboxymethyl-amino)-acetic acid

125 mg (1.1 mmol) of chloroacetic acid chloride was added at −20° C. to a solution of 0.51 g (1 mmol) of 3 and 606 mg (6 mmol) of triethylamine in 10 ml of DMF. The reaction solution was stirred for one hour at room temperature. The solution was poured into vigorously-stirred diethyl ether. The precipitate was filtered off, taken up in water and immediately freeze-dried. The substance was checked for purity by HPLC:HyPurity C18 (5 μm, 150×3.0 mm) with acetonitrile-:water-:trifluoroacetic acid gradient (3:96.9:0.1→99.9:0:0.1). The desired product 8 was produced with a yield of 82% (520 mg, 820 μmol).

MS-FAB: 590 (M⁺+1.31)

### EXAMPLE 9

### (S,S)-({2-{[2-(Bis-carboxymethyl-amino)-propyl]-carboxymethyl-amino}-1-[4-(2,5-dioxo-2,5-dihydro-pyrrol-1-yl)-benzyl]-ethyl}-carboxymethyl-amino)-acetic acid

In a way similar to Tetrahedron Lett.; 38; 46; 1997; 8089-8092:

A mixture that consists of about 1 g of dried silica gel, 100 mg (1.0 mmol) of maleic acid anhydride, 512 mg (1.0 mmol) of 3 and 35.6 mg (0.1 mmol) of tantalum(V) chloride was heated in a microwave (300 W) for 5 minutes. The residue was eluted on a frit with methanol. The filtrate was concentrated by evaporation. The residue was taken up in water. The solution was freeze-dried. A portion of the residue was purified by semi-preparative RP-HPLC. Acetonitrile-water mixture (20:80). The substance was checked for purity by HPLC: HyPurity C18 (5 □m, 150×3.0 mm) with acetonitrile-:water-:trifluoroacetic acid gradient (3:96.9:0.1→99.9:0:0.1). The desired product 9 was produced with 94 mg (0.16 mmol) in 96% purity.

MS-FAB: 593 (M⁺+1.42)

### EXAMPLE 10

### Antibody conjugate of Example 4, namely (S,S)-[(2-{[2-(bis-carboxymethyl-amino)-propyl]-carboxymethyl-amino}-1-{4-[3-(2,5-dioxo-2,5-dihydro-pyrrol-1-yl)-propionylamino]-benzyl}-ethyl)-carboxymethyl-amino]-acetic acid

200 μg of an antibody with freely accessible thiol groups (e.g., HuM195 (cf. Michael R. McDevitt, J. Nuc. Med. 40, 1999, 1722; commercially available at Protein Design Labs Inc., Mountainview, Calif., USA)—if the antibody has no freely accessible thiol groups, the latter can be produced by the use of 2-iminothiolane HCl (e.g., EP 0 607 222 B1))—was diluted in 1.2 ml of borate buffer (50 mmol, pH 8.5), mixed with 159 μg (240 nmol) of product from Example 4, dissolved in 50 μl of borate buffer (see above), and stirred for 3 hours at 37° C. It was purified on a NAP-5-column (Amersham Pharmacia Biotech AB, Sephadex G-25, Mobile Phase: PBS).

### EXAMPLE 11

### (S,S)-{[2-{[2-(Bis-carboxymethyl-amino)-propyl]-carboxymethyl-amino}-1-(4-{3-[2-(2,5-dioxo-2,5-dihydro-pyrrol-1-yl)-ethyl]-thioureido}-benzyl)-ethyl]-carboxymethyl-amino}-acetic acid

A solution of 154 mg (1.1 mmol) of 1-(2-amino-ethyl)-pyrrole-2,5-dione in 2 ml of dioxane was added in drops to a stirred solution of 555 mg (1 mmol) of 5 in 5 ml of DMF. The reaction solution was stirred overnight and added drop by drop to 80 ml of vigorously stirred diethyl ether. The precipitate was filtered off and taken up in water. The solution was freeze-dried. A portion of the residue was purified by semi-preparative RP-HPLC. Acetonitrile-water mixture (20:80). The substance was checked for purity by HPLC: HyPurity C18 (5 μm, 150×3.0 mm) with acetonitrile-:water-:trifluoroacetic acid gradient (3:96.9:0.1→99.9:0:0.1). The desired product 11 was produced with 0.104 g (0.15 mmol) in high purity.

MS-FAB: 696 (M⁺+1.33)

### EXAMPLE 12

### Indium complex of the antibody conjugate of Example 4, namely (S,S)-[(2-{[2-(bis-carboxymethyl-amino)-propyl]-carboxymethyl-amino}-1-{4-[3-(2,5-dioxo-2,5-dihydro-pyrrol-1-yl)-propionylamino]-benzyl}-ethyl)-carboxymethyl-amino]-acetic acid

200 μg of an antibody with freely accessible thiol groups (e.g., HuM195 (cf. Michael R. McDevitt, J. Nuc. Med. 40, 1999, 1722; commercially available at Protein Design Labs Inc., Mountainview, Calif., USA)—if the antibody has no freely accessible thiol groups, the latter can be produced by the use of 2-iminothiolane HCl (e.g., EP 0 607 222 B1))—was diluted in 1.2 ml of borate buffer (50 mmol, pH 8.5), mixed with 159 μg (240 nmol) of product of Example 11, dissolved in 50 μl of borate buffer (see above), and stirred for 3 hours at 37° C. The borate-buffer solution was exchanged for an acetate buffer by the sample solution being set at 0.1 M (pH 6.0) three times for 1 hour in the Slide-A-Lyzer 10000, Pierce, MWCO (dialysis process) against 200 ml of NaOAc buffer in each case. Then, it was set at 0.1 M (pH 6) overnight against 400 ml of NaOAc buffer. The solution was mixed with 80 μl (0.05M HCl) of [¹¹¹In]InCl₃ (27.88 MBq) and stirred for 30 minutes at room temperature. It was purified on a NAP-5 column (Amersham Pharmacia Biotech AB, Sephadex G-25, Mobile Phase: PBS).

### EXAMPLE 13

### Yttrium complex of the antibody conjugate of (1″R,2R,5S)-5-({[2-({1-carboxy-5-[3-(2,5-dioxo-2,5-dihydro-pyrrol-1-yl)-propionylamino]-pentyl}-carboxymethyl-amino)-ethyl]-carboxymethyl-amino}-methyl)-1-carboxymethyl-pyrrolidine-2-carboxylic acid

200 μg of an antibody with freely accessible thiol groups (e.g., HuM195 (cf. Michael R. McDevitt, J. Nuc. Med. 40, 1999, 1722; commercially available at Protein Design Labs Inc., Mountainview, Calif., USA)—if the antibody has no freely accessible thiol groups, the latter can be produced by the use of 2-iminothiolane HCl (e.g., EP 0 607 222 B1))—was diluted in 1.2 ml of borate buffer (50 mmol, pH 8.5), mixed with 159 μg (240 nmol) of product from Example 11, dissolved in 50 μl of borate buffer (see above), and stirred for 3 hours at 37° C. The borate-buffer solution was exchanged for an acetate buffer by the sample solution being set at 0.1 M (pH 6.0) three times for 1 hour in the Slide-A-Lyzer 10000, Pierce, MWCO (dialysis process) against 200 ml of NaOAc buffer in each case. Finally, it was set at 0.1 M (pH 6) overnight against 400 ml of NaOAc buffer. The solution was mixed with 50 MBq of [⁹⁰Y]YCl₃ and stirred for 30 minutes at room temperature. It was purified on a NAP-5 column (Amersham Pharmacia Biotech AB, Sephadex G-25, Mobile Phase: PBS).

### EXAMPLE 14

### Scandium complex of the antibody conjugate of (1″R,2R,5S)-5-({[2-({1-carboxy-5-[3-(2,5-dioxo-2,5-dihydro-pyrrol-1-yl)-propionylamino]-pentyl}-carboxymethyl-amino)-ethyl]-carboxymethyl-amino}-methyl)-1-carboxymethyl-pyrrolidine-2-carboxylic acid

200 μg of an antibody with freely accessible thiol groups (e.g., HuM195 (cf. Michael R. McDevitt, J. Nuc. Med. 40, 1999, 1722; commercially available at Protein Design Labs Inc., Mountainview, Calif., USA)—if the antibody has no freely accessible thiol groups, the latter can be produced by the use of 2-iminothiolane HCl (e.g., EP 0 607 222 B1))—was diluted in 1.2 ml of borate buffer (50 mmol, pH 8.5), mixed with 159 μg (240 nmol) of product from Example 11, dissolved in 50 μl of borate buffer (see above), and stirred for 3 hours at 37° C. The borate-buffer solution was exchanged for an acetate buffer by the sample solution being set at 0.1 M (pH 6.0) three times for 1 hour in the Slide-A-Lyzer 10000, Pierce, MWCO (dialysis process) against 200 ml of NaOAc buffer in each case. Finally, it was set at 0.1 M (pH 6) overnight against 400 ml of NaOAc buffer. The solution was mixed with 50 MBq of [⁴⁷Sc]ScCl₃ and stirred for 30 minutes at room temperature. It was purified on a NAP-5 column (Amersham Pharmacia Biotech AB, Sephadex G-25, Mobile Phase: PBS).

### EXAMPLE 15

### Lutetium complex of the antibody conjugate of (1′R,2R,5S)-5-({[2-({1-carboxy-5-[3-(2,5-dioxo-2,5-dihydro-pyrrol-1-yl)-propionylamino]-pentyl}-carboxymethyl-amino)-ethyl]-carboxymethyl-amino}-methyl)-1-carboxymethyl-pyrrolidine-2-carboxylic acid

200 μg of an antibody with freely accessible thiol groups (e.g., HuM195 (cf. Michael R. McDevitt, J. Nuc. Med. 40, 1999, 1722; commercially available at Protein Design Labs Inc., Mountainview, Calif., USA)—if the antibody has no freely accessible thiol groups, the latter can be produced by the use of 2-iminothiolane HCl (e.g., EP 0 607 222 B1))—was diluted in 1.2 ml of borate buffer (50 mmol, pH 8.5), mixed with 159 μg (240 nmol) of product from Example 11, dissolved in 50 μl of borate buffer (see above), and stirred for 3 hours at 37° C. The borate-buffer solution was exchanged for an acetate buffer by the sample solution being set at 0.1 M (pH 6.0) three times for 1 hour in the Slide-A-Lyzer 10000, Pierce, MWCO (dialysis process) against 200 ml of NaOAc buffer in each case. Finally, it was set at 0.1 M (pH 6) overnight against 400 ml of NaOAc buffer. The solution was mixed with 50 MBq of [¹⁷⁷Lu]LuCl₃ and stirred for 30 minutes at room temperature. It was purified on a NAP-5 column (Amersham Pharmacia Biotech AB, Sephadex G-25, Mobile Phase: PBS).

### EXAMPLE 16

### a) (S)-6-Benzyloxycarbonylamino-2-(2-tert-butoxycarbonylamino-acetylamino)-hexanoic acid benzyl ester

1.55 g (13.5 mmol) of N-hydroxysuccinimide was added to a stirred solution of 2.15 g (12.28 mmol) of Boc-Gly-OH and 4.08 ml (29.5 mmol) of triethylamine in 50 ml of dichloromethane. After 20 minutes, 5 g (12.3 mmol) of H-Lys-(Z)-OBzl hydrochloride, which was dissolved in some dichloromethane, and 2.78 g (13.5 mmol) of dicyclohexylcarbodiimide in some dichloromethane were added. The solution was stirred for three days and poured into 250 ml of ice water. The organic phase was separated. The aqueous phase was extracted several times with dichloromethane. The combined organic phases were washed with aqueous, saturated sodium chloride solution, dried with sodium sulfate and concentrated by evaporation. The residue was purified by column chromatography (SiO₂ hexane:ethyl acetate 4:1-3 hexane:ethyl acetate 1:1). The desired product 16a was produced in a yield of 96% (6.2 g; 11.7 mmol).

MS-FAB: 528 (M⁺+1.75)

### b) (S)-6-Benzyloxycarbonylamino-2-(2-{2-[2-(2-tert-butoxycarbonylamino-acetylamino)-acetylamino]-acetylamino}-acetylamino)-hexanoic acid benzyl ester

30 ml of trifluoroacetic acid was added drop by drop at 0° C. to a solution of 6.2 g (11.7 mmol) of 16a in 30 ml of dichloromethane. The solution was stirred for 2 hours at room temperature and concentrated by evaporation in a rotary evaporator. 50 ml of water and 50 ml of toluene were added and again removed in a rotary evaporator. The last operating step was repeated three times. Finally, it was concentrated at the oil pump.

A stirred solution of the residue in 90 ml of dichloromethane was added at 0° C. with 2.37 g (18.3 mmol) of diisopropylethyldiamine, 3.02 g (14.7 mmol) of dicyclohexylcarbodiimide, dissolved in a little dichloromethane, 1.69 g (14.7 mmol) of N-hydroxysuccinimide and 3.4 g (14.7 mmol) of Boc-Gly-Gly-OH. The suspension was stirred for three days at room temperature and mixed with 150 ml of ice water. The organic phase was separated. The aqueous phase was extracted several times with ethyl acetate. The combined organic phases were washed with aqueous, saturated sodium chloride solution, dried with sodium sulfate and concentrated by evaporation. The residue was purified by column chromatography (SiO₂ ethyl acetate→methanol:ethyl acetate 15:85). The desired product 16b was produced in a yield of 53% (4.18 g; 6.5 mmol).

MS-FAB: 643 (M⁺+1.56)

### c) (S)-2-{2-[2-(2-tert-Butoxycarbonylamino-acetylamino)-acetylamino]-acetylamino}-6-[3-(2,5-dioxo-2,5-dihydro-pyrrol-1-yl)-propionylamino]-hexanoic acid

1 g of Pd/C (10%) was added to a solution of 4.18 g (6.5 mmol) of 16b in 40 ml of isopropanol. The atmosphere over the stirred solution was saturated with hydrogen. The reaction solution was stirred for 90 minutes, filtered and concentrated by evaporation.

The residue was mixed at 0° C. with 20 ml of DMF, 1.5 g (12 mmol) of diisopropylethylamine and 2.4 mg (9 mmol) of 3-(2,5-dioxo-2,5-dihydro-pyrrol-1-yl)-propionic acid 2,5-dioxo-pyrrolidin-1-yl ester (Aldrich). The reaction solution was stirred for 4 hours at room temperature. The solution was added to HCl solution (pH 4) and extracted several times with ethyl acetate. The combined organic phases were washed with aqueous, saturated sodium chloride solution, dried with sodium sulfate and concentrated by evaporation. The residue was purified by column chromatography (SiO₂ ethyl acetate→methanol:ethyl acetate 20:80). The desired product 16c was produced in a yield of 67% (2.5 g; 4.4 mmol).

MS-FAB: 570 (M⁺+1.31)

### d) (1S,1′S,4S)-1-[4-{3-[({[({1-Carboxy-5-[3-(2,5-dioxo-2,5-dihydro-pyrrol-1-yl)-propionylamino]-pentylcarbamoyl}-methyl)-carbamoyl]-methyl}-carbamoyl)-methyl]-thioureido}]-benzyl-4-methyl-DTPA

5 ml of trifluoroacetic acid was added drop by drop at 0° C. to a stirred solution of 570 mg (1 mmol) of 16c in 5 ml of dichloromethane. The solution was stirred for 2 hours at room temperature and concentrated by evaporation in a rotary evaporator. The residue was absorptively precipitated with diethyl ether. Finally, it was concentrated at the oil pump.

The residue was taken up in DMF and mixed with ˜200 mg (˜2 mmol) of triethylamine and 505 mg (0.9 mmol) of 5. The solution was stirred for 3 hours at 40° C. and added in drops to vigorously stirred diethyl ether. The precipitate was filtered off and purified by RP-column chromatography.

MS-FAB: 1016 (M⁺+1.31)

### EXAMPLE 17

### Gadolinium complex of (S,S)-[(1-(4-amino-benzyl)-2-{[2-(bis-carboxymethyl-amino)-propyl]-carboxymethyl-amino}-ethyl)-carboxymethyl-amino]-acetic acid

128.1 mg (0.25 mmol) of 3 was suspended in 4 ml of distilled water, heated to 80° C. and brought into solution. It was mixed in portions with 45.3 mg (0.125 mmol) of Gd₂O₃. The suspension was heated to 80° C. and stirred for one hour. The solution was cooled to room temperature and set at pH=7 with sodium hydroxide solution (1 M). The water was removed by freeze-drying. The desired product was produced with a yield of 166.6 mg (0.25 mmol, 99.8%).

MS-FAB (M⁺+1.21): 668

### EXAMPLE 18

### Antibody conjugate of (1S,1′S,4S)-1-[4-{3-[({[{1-carboxy-5-[3-(2,5-dioxo-2,5-dihydro-pyrrol-1-yl)-propionylamino]-pentylcarbamoyl}-methyl)-carbamoyl]-methyl}-carbamoyl)-methyl]-thioureido}]-benzyl-4-methyl-DTPA

200 μs of an antibody with freely accessible thiol groups (e.g., HuM195 (cf. Michael R. McDevitt, J. Nuc. Med. 40, 1999, 1722; commercially available at Protein Design Labs Inc., Mountainview, Calif., USA)—if the antibody has no freely accessible thiol groups, the latter can be produced by the use of 2-iminothiolane HCl (e.g., EP 0 607 222 B1))—was diluted in 1.2 ml of borate buffer (50 mmol, pH 8.5), mixed with 243 μg (240 nmol) of product from Example 16d, dissolved in 50 μl of borate buffer (see above), and stirred for 3 hours at 37° C. It was purified on a NAP-5 column (Amersham Pharmacia Biotech AB, Sephadex G-25, Mobile Phase: PBS).

### EXAMPLE 19

### Yttrium complex of the antibody conjugate of (1S,1′S,4S)-1-[4-{3-[({[({1-carboxy-5-[3-(2,5-dioxo-2,5-dihydro-pyrrol-1-yl)-propionylamino]-pentylcarbamoyl}-methyl)-carbamoyl]-methyl}-carbamoyl)-methyl]-thioureido}-benzyl-4-methyl-DTPA

200 μg of an antibody with freely accessible thiol groups (e.g., HuM195 (cf. Michael R. McDevitt, J. Nuc. Med. 40, 1999, 1722; commercially available at Protein Design Labs Inc., Mountainview, Calif., USA)—if the antibody has no freely accessible thiol groups, the latter can be produced by the use of 2-iminothiolane HCl (e.g., EP 0 607 222 B1))—was diluted in 1.2 ml of borate buffer (50 mmol, pH 8.5), mixed with 243 μg (240 nmol) of product of Example 16d, dissolved in 50 μl of borate buffer (see above), and stirred for 3 hours at 37° C. The borate-buffer solution was exchanged for an acetate buffer by the sample solution being set at 0.1 M (pH 6.0) three times for 1 hour in the Slide-A-Lyzer 10000, Pierce, MWCO (dialysis process) against 200 ml of NaOAc buffer in each case. Finally, it was set at 0.1 M (pH 6) overnight against 400 ml of NaOAc buffer. The solution was mixed with 50 MBq of [⁹⁰Y]YCl₃ and stirred for 30 minutes at room temperature. It was purified on a NAP-5 column (Amersham Pharmacia Biotech AB, Sephadex G-25, Mobile Phase: PBS).

### EXAMPLE 20

### Scandium complex of the antibody conjugate (1S,1′S,4S)-1-[4-{3-[({[({1-carboxy-5-[3-(2,5-dioxo-2,5-dihydro-pyrrol-1-yl)-propionylamino]-pentylcarbamoyl}-methyl)-carbamoyl]-methyl}-carbamoyl)-methyl]-thioureido}]-benzyl-4-methyl-DTPA

200 μg of an antibody with freely accessible thiol groups (e.g., HuM195 (cf. Michael R. McDevitt, J. Nuc. Med. 40, 1999, 1722; commercially available at Protein Design Labs Inc., Mountainview, Calif., USA)—if the antibody has no freely accessible thiol groups, the latter can be produced by the use of 2-iminothiolane HCl (e.g., EP 0 607 222 B1))—was diluted in 1.2 ml of borate buffer (50 mmol, pH 8.5), mixed with 243 μg (240 nmol) of product from Example 16d, dissolved in 50 μl of borate buffer (see above), and stirred for 3 hours at 37° C. The borate-puffer solution was exchanged for an acetate buffer by the sample solution being set at 0.1 M (pH 6.0) three times for 1 hour in the Slide-A-Lyzer 10000, Pierce, MWCO (dialysis process) against 200 ml of NaOAc buffer in each case. Finally, it was set at 0.1 M (pH 6) overnight against 400 ml of NaOAc buffer. The solution was mixed with 50 MBq of [⁴⁷Sc]ScCl₃ and stirred for 30 minutes at room temperature. It was purified on a NAP-5 column (Amersham Pharmacia Biotech AB, Sephadex G-25, Mobile Phase: PBS).

### EXAMPLE 21

### Lutetium complex of the antibody conjugate (1S,1′S,4S)-1-[4-{3-[({[({1-carboxy-5-[3-(2,5-dioxo-2,5-dihydro-pyrrol-1-yl)-propionylamino]-pentylcarbamoyl}-methyl)-carbamoyl]-methyl}-carbamoyl)-methyl]-thioureido}]-benzyl-4-methyl-DTPA

200 μg of an antibody with freely accessible thiol groups (e.g., HuM195 (cf. Michael R. McDevitt, J. Nuc. Med. 40, 1999, 1722; commercially available at Protein Design Labs Inc., Mountainview, Calif., USA)—if the antibody has no freely accessible thiol groups, the latter can be produced by the use of 2-iminothiolane HCl (e.g., EP 0 607 222 B1))—was diluted in 1.2 ml of borate buffer (50 mmol, pH 8.5), mixed with 243 μg (240 nmol) of product from Example 16d, dissolved in 50 μl of borate buffer (see above), and stirred for 3 hours at 37° C. The borate-buffer solution was exchanged for an acetate buffer by the sample solution being set at 0.1 M (pH 6.0) three times for 1 hour in the Slide-A-Lyzer 10000, Pierce, MWCO (dialysis process) against 200 ml of NaOAc buffer in each case. Finally, it was set at 0.1 M (pH 6) overnight against 400 ml of NaOAc buffer. The solution was mixed with 50 MBq of [¹⁷⁷Lu]LuCl₃ and stirred for 30 minutes at room temperature. It was purified on a NAP-5 column (Amersham Pharmacia Biotech AB, Sephadex G-25, Mobile Phase: PBS).

### EXAMPLE 22

### (R,R)-{[2-{[2-(Bis-carboxymethyl-amino)-propyl]-carboxymethyl-amino}-1-(4-nitro-benzyl)-ethyl]-carboxymethyl-amino}-acetic acid

The synthesis of Example 22 is carried out analogously to Example 1, with the difference that not (S)- but rather (R)-2-amino-1-propanol is used, and also not (S)- but rather (R)-2-tert-butoxycarbonylamino-3-(4-nitro-phenyl)-propionic acid was used as a component. The desired product 22 was produced with a yield in high purity (>96%, RP-HPLC).

MS-FAB: 513 (M⁺+1.42)

### EXAMPLE 23

### (R,R))-[(1-(4-Amino-benzyl)-2-{[2-(bis-carboxymethyl-amino)-propyl]-carboxymethyl-amino}-ethyl)-carboxymethyl-amino]-acetic acid

The synthesis of Example 23 is carried out analogously to Example 3, Method A, with the difference that as an educt, not 1h, but rather 22 was used. The desired product 23 was produced with a yield of 96%.

MS-FAB: 513 (M⁺+1.42)

### EXAMPLE 24

### (R,R))-{[2-(4-Amino-phenyl)-1-({[2-(bis-tert-butoxycarbonylmethyl-amino)- propyl]-tert-butoxycarbonylmethyl-amino}-methyl)-ethyl]-tert-butoxycarbonylmethyl-amino}-acetic acid tert-butyl ester

The synthesis of Example 24 is carried out analogously to Example 2, with the difference that as an educt, not 1g but rather the corresponding precursor from the synthesis of 22 was used. The desired product 24 was produced with a yield of 86%.

MS-FAB: 794 (M⁺+1.53)

### EXAMPLE 25

### (R,R)-[(1-(4-Isothiocyanato-benzyl)-2-{[2-(bis-carboxymethyl-amino)-propyl]-carboxymethyl-amino}-ethyl)-carboxymethyl-amino]-acetic acid

The synthesis of Example 25 is carried out analogously to Example 5, with the difference that as educt, not 3, but rather 23 was used. The desired product 25 was produced with a yield of 74%.

MS-FAB: 555 (M⁺+1.33)

### EXAMPLE 26

### (R,R)-({2-{[2-(Bis-carboxymethyl-amino)-propyl]-carboxymethyl-amino}-1-[4-(2-bromo-acetylamino)-benzyl]-ethyl}-carboxymethyl-amino)-acetic acid

The synthesis of Example 26 is carried out analogously to Example 6, with the difference that as educt, not 3, but rather 23 was used. The desired product 23 was produced with a yield of 71%.

MS-FAB: 590 (M⁺+1.48)

### EXAMPLE 27

### a) N-[4-(2-(Bis-carboxymethyl-amino)-3-{[2-(bis-carboxymethyl-amino)-propyl]-carboxymethyl-amino}-propyl)-phenyl]-succinaminic acid

200 mg (2 mmol) of succinic acid anhydride was added to a solution of 793 mg (1 mmol) of 24 and 0.28 ml (˜2 mmol) of diisopropylethylamine in 20 ml THF. The solution was stirred for 2 hours at room temperature. The solution was concentrated by evaporation to one third of the volume, diluted with dichloromethane and added to an aqueous solution (pH=4.5). The aqueous phase was separated and extracted several times with dichloromethane. The combined organic phases were washed with sodium chloride solution. It was dried with sodium sulfate and concentrated by evaporation in a rotary evaporator. The residue was purified by column chromatography (CH₂Cl₂—MeOH). The desired product 27a was produced with a yield of 85% (0.85 mmol).

MS-FAB: 613 (M⁺+1.58)

### b) (1R,1′S,4R)-1-[4-(3-{[({[({1-Carboxy-5-[3-(2,5-dioxo-2,5-dihydro-pyrrol-1-yl)- propionylamino]-pentylcarbamoyl}-methyl)-carbamoyl]-methyl}-carbamoyl)-methyl]-carbamoyl}-propionyl)]-benzyl-4-methyl-DTPA

5 ml of trifluoroacetic acid was added drop by drop at 0° C. to a stirred solution of 570 mg (1 mmol) of 16c in 5 ml of dichloromethane. The solution was stirred for 2 hours at room temperature and concentrated by evaporation in a rotary evaporator. The residue was absorptively precipitated with diethyl ether. Finally, it was concentrated at the oil pump. The residue was suspended in dichloromethane, and mixed with 0.25 g (˜2 mmol) of Hünig base, 304 mg (2 mmol) of 1-hydroxybenzotriazole ˜H₂O (HOBT) and 122 mg (2 mmol) of 27a. The solution was cooled to 0° C. and mixed with 419 mg (21 mmol) of 1-(dimethylaminopropyl)-3-ethylcarbodiimide (EDCI). The solution was stirred for 12 hours and poured onto ice water. The aqueous phase was separated and extracted several times with dichloromethane. The combined organic phases were washed with sodium chloride solution. It was dried with sodium sulfate and concentrated by evaporation in a rotary evaporator. The residue was purified by column chromatography (CH₂Cl₂-acetonitrile). The desired product was produced with a yield of 85% (0.85 mmol), which then was taken up in 5 ml of dichloromethane and 3 ml of anisole and was mixed with 5 ml of trifluoroacetic acid at 0° C. The solution was stirred for 8 hours, concentrated by evaporation and absorptively precipitated with diethyl ether. The desired product 27b was produced in a yield of 90% (813.9 mg).

MS-FAB: 1064 (M⁺+1.38)

### EXAMPLE 28

### Antibody conjugate of (1R,1′S,4R)-1-[4-(3-{[({[({1-carboxy-5-[3-(2,5-dihydro-pyrrol-1-yl)-propionylamino]-pentylcarbamoyl}-methyl)-carbamoyl]-methyl}-carbamoyl)-methyl]-carbamoyl}-propionyl)1-benzyl-4-methyl-DTPA

200 μg of an antibody with freely accessible thiol groups (e.g., HuM195 (cf. Michael R. McDevitt, J. Nuc. Med. 40, 1999, 1722; commercially available at Protein Design Labs Inc., Mountainview, Calif., USA)—if the antibody has no freely accessible thiol groups, the latter can be produced by the use of 2-iminothiolane HCl (e.g., EP 0 607 222 B1))—was diluted in1.2 ml of borate buffer (50 mmol, pH 8.5), mixed with 255 μg (240 nmol) of product from Example 27b, dissolved in 50 μl of borate buffer (see above), and stirred for 3 hours at 37° C. It was purified on a NAP-5 column (Amersham Pharmacia Biotech AB, Sephadex G-25, Mobile Phase: PBS).

### EXAMPLE 29

### Yttrium complex of the antibody conjugate of (1R,1′S,4R)-1-[4-(3-{[({[({1-carboxy-5-[3-(2,5-dioxo-2,5-dihydro-pyrrol-1-yl)-propionylamino]-pentylcarbamoyl}-methyl)-carbamoyl]-methyl}-carbamoyl)-methyl]-carbamoyl}-propionyl)]-benzyl-4-methyl-DTPA

200 μg of an antibody with freely accessible thiol groups (e.g., HuM195 (cf. Michael R. McDevitt, J. Nuc. Med. 40, 1999, 1722; commercially available at Protein Design Labs Inc., Mountainview, Calif., USA)—if the antibody has no freely accessible thiol groups, the latter can be produced by the use of 2-iminothiolane HCl (e.g., EP 0 607 222 B1))—was diluted in 1.2 ml of borate buffer (50 mmol, pH 8.5), mixed with 255 μg (240 nmol) of product from Example 27b, dissolved in 50 μl of borate buffer (see above), and stirred for 3 hours at 37° C. The borate-buffer solution was exchanged for an acetate buffer by the sample solution being set at 0.1 M (pH 6.0) three times for 1 hour in the Slide-A-Lyzer 10000, Pierce, MWCO (dialysis process) against 200 ml of NaOAc buffer in each case. Finally, it was set at 0.1 M (pH 6) overnight against 400 ml of NaOAc buffer. The solution was mixed with 50 MBq of [⁹⁰Y]YCl₃ and stirred for 30 minutes at room temperature. It was purified on a NAP-5 column (Amersham Pharmacia Biotech AB, Sephadex G-25, Mobile Phase: PBS).

### EXAMPLE 30

### Lutetium complex of the antibody conjugate of (1R,1′S,4R)-1-[4-(3-{[({[({1-carboxy-5-[3-(2,5-dioxo-2,5-dihydro-pyrrol-1-yl)-propionylamino]- pentylcarbamoyl}-methyl)-carbamoyl]-methyl}-carbamoyl)-methyl]-carbamoyl}-propionyl)]-benzyl-4-methyl-DTPA

200 μg of an antibody with freely accessible thiol groups (e.g., HuM195 (cf. Michael R. McDevitt, J. Nuc. Med. 40, 1999, 1722; commercially available at Protein Design Labs Inc., Mountainview, Calif., USA)—if the antibody has no freely accessible thiol groups, the latter can be produced by the use of 2-iminothiolane HCl (e.g., EP 0 607 222 B1))—was diluted in 1.2 ml of borate buffer (50 mmol, pH 8.5), mixed with 255 μg (240 nmol) of product from Example 27b, dissolved in 50 μof borate buffer (see above), and stirred for 3 hours at 37° C. The borate-buffer solution was exchanged for an acetate buffer by the sample solution being set at 0.1 M (pH 6.0) three times for 1 hour in the Slide-A-Lyzer 10000, Pierce, MWCO (dialysis process) against 200 ml of NaOAc buffer in each case. Finally, it was set at 0.1 M (pH 6) overnight against 400 ml of NaOAc buffer. The solution was mixed with 50 MBq of [¹⁷⁷Lu]LuCl₃ and stirred for 30 minutes at room temperature. It was purified on a NAP-5 column (Amersham Pharmacia Biotech AB, Sephadex G-25, Mobile Phase: PBS).

### EXAMPLE 31

### Scandium complex of the antibody conjugate of (1R,1′S,4R)-1-[4-(3-{[({[({1-carboxy-5-[3-(2,5-dioxo-2,5-dihydro-pyrrol-1-yl)-propionylamino]-pentylcarbamoyl}-methyl)-carbamoyl]-methyl}-carbamoyl)-methyl]-carbamoyl}-propionyl)]-benzyl-4-methyl-DTPA

200 μg of an antibody with freely accessible thiol groups (e.g., HuM195 (cf. Michael R. McDevitt, J. Nuc. Med. 40, 1999, 1722; commercially available at Protein Design Labs Inc., Mountainview, Calif., USA)—if the antibody has no freely accessible thiol groups, the latter can be produced by the use of 2-iminothiolane HCl (e.g., EP 0 607 222 B1))—was diluted in 1.2 ml of borate buffer (50 mmol, pH 8.5), mixed with 255 μg (240 nmol) of product from Example 27b, dissolved in 50 μl of borate buffer (see above), and stirred for 3 hours at 37° C. The borate-buffer solution was exchanged for an acetate buffer by the sample solution being set at 0.1 M (pH 6.0) three times for 1 hour in the Slide-A-Lyzer 10000, Pierce, MWCO (dialysis process) against 200 ml of NaOAc buffer in each case. Finally, it was set at 0.1 M (pH 6) overnight against 400 ml of NaOAc buffer. The solution was mixed with 50 MBq of [⁴⁷Sc]ScCl₃ and stirred for 30 minutes at room temperature. It was purified on a NAP-5 column (Amersham Pharmacia Biotech AB, Sephadex G-25, Mobile Phase: PBS).

Without further elaboration, it is believed that one skilled in the art can, using the preceding description, utilize the present invention to its fullest extent. The preceding preferred specific embodiments are, therefore, to be construed as merely illustrative, and not limitative of the remainder of the disclosure in any way whatsoever.

In the foregoing and in the examples, all temperatures are set forth uncorrected in degrees Celsius and, all parts and percentages are by weight, unless otherwise indicated.

The entire disclosures of all applications, patents and publications, cited herein and of corresponding German application No. 10305463.4, filed Feb. 4, 2003, and U.S. Provisional Application Ser. No. 60/446,538, filed Feb. 12, 2003 are incorporated by reference herein.

The preceding examples can be repeated with similar success by substituting the generically or specifically described reactants and/or operating conditions of this invention for those used in the preceding examples.

From the foregoing description, one skilled in the art can easily ascertain the essential characteristics of this invention and, without departing from the spirit and scope thereof, can make various changes and modifications of the invention to adapt it to various usages and conditions.

1. Conjugates of general formulas VIIa and VIIb: 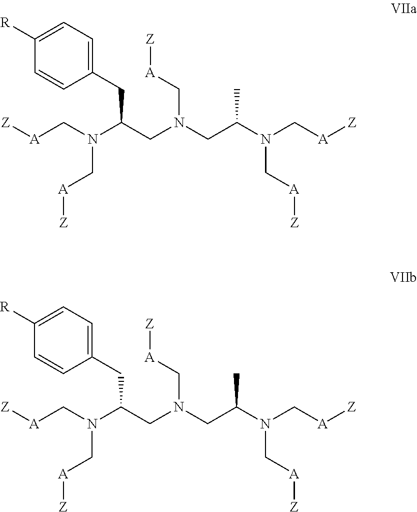
in which
Z stands for a hydrogen atom or a metal ion equivalent of an element of atomic numbers 21-29, 31, 32, 37-39, 42-44, 46, 47, 49, 58-71, 75, 77, 82 or 83,
A stands for a group —COO—,
R stands for a nitro group, an amino group, or another functional group which can be linked with a biomolecule, or for a straight-chain or branched, saturated or unsaturated C₁-C₂₅-alkyl radical that is optionally interrupted by one to six O atoms or phenylene,
—NHCO—, —CONH—, 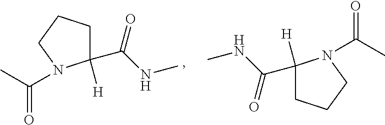
and/or —NH—(C═S)—NH— groups and that optionally is substituted at any location with one to six carboxyl groups, hydroxyl groups, amino groups or other functional groups, as well as their salts with organic or inorganic bases, provided that the alkyl radical contains at least one functional group, which can be linked with a biomolecule, and that at least two Z stand for a metal ion equivalent.

2. Compounds according to claim 1, in which at least two of radicals Z stand for a metal ion equivalent of a paramagnetic element of atomic numbers 21-29, 42, 44 and 58-70.

3. Compounds according to claim 1, in which at least two of radicals Z stand for a metal ion equivalent of a radioactive element of atomic numbers 26, 27, 29, 31, 32, 37-39, 43, 46, 47, 49, 61, 62, 64, 70, 71, 75, 77, 82 and 83.

4. Compounds according to claim 1, in which R stands for a radical

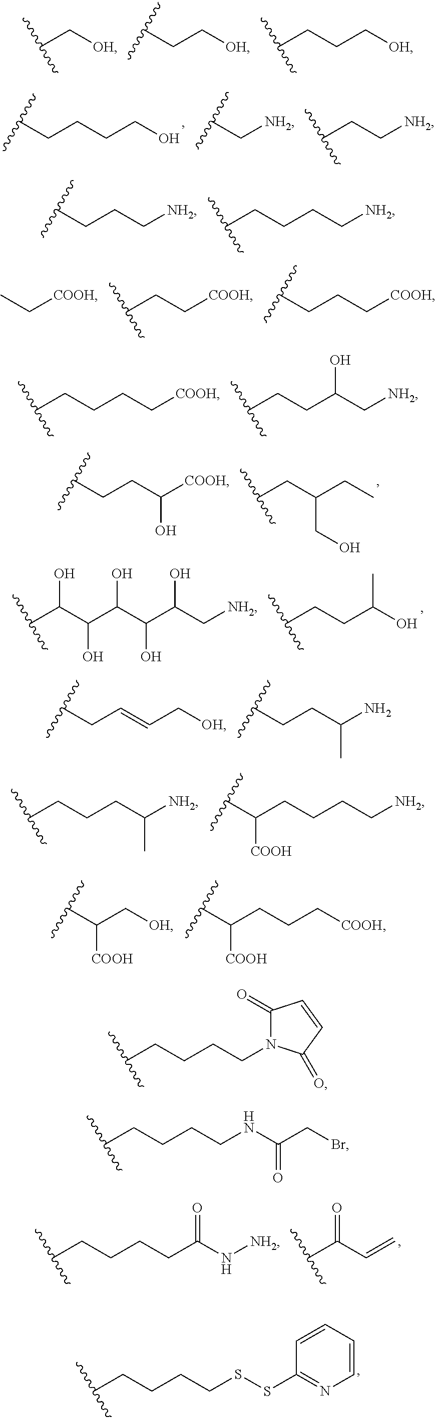

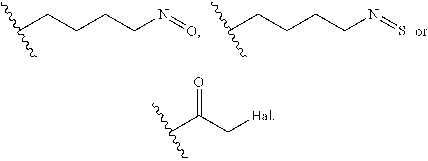

5. Compounds according to claim 1, in which R stands for one a radical

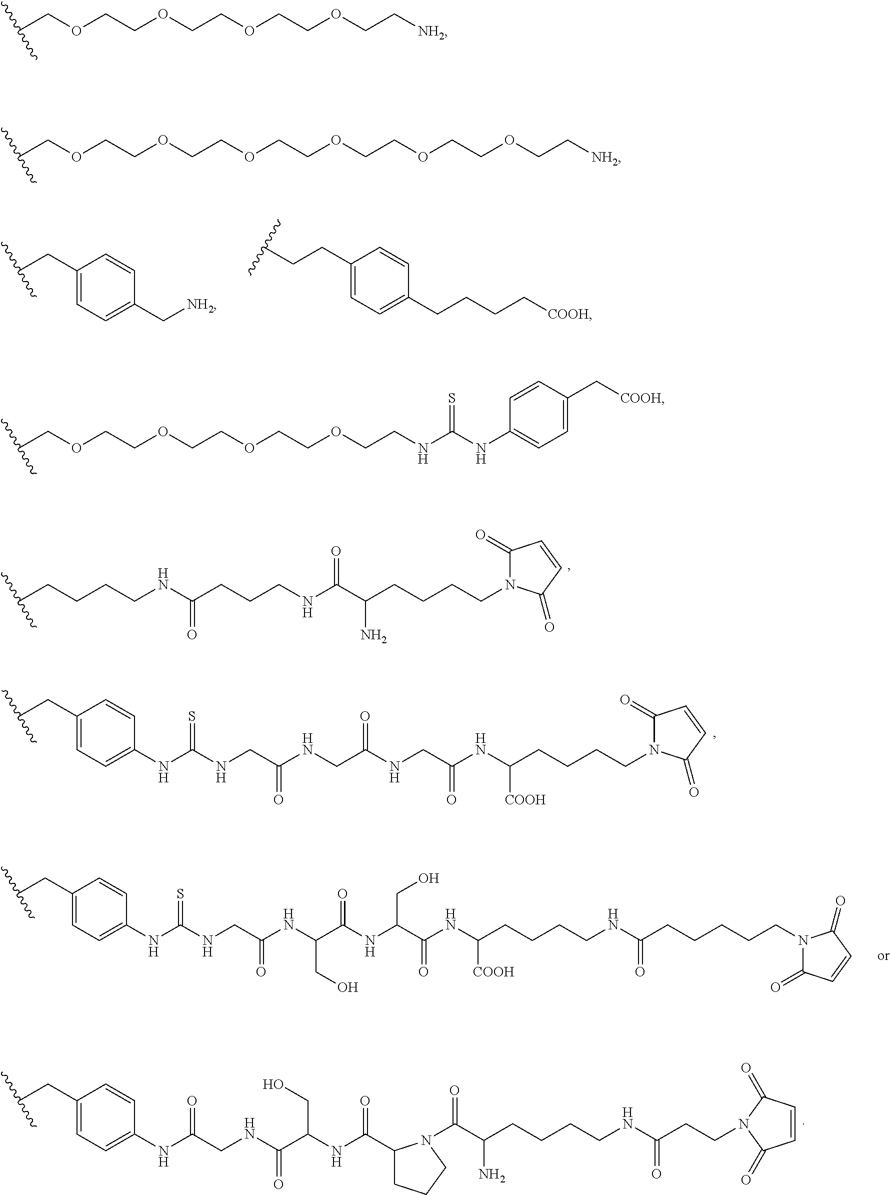

6. Compounds according to claim 1, in which R stands for a functional group carboxyl, activated carboxyl, amino, nitro, isocyanate, isothiocyanate, hydrazine, semicarbazide, thiosemibarbazide, chloroacetamide, bromoacetamide, iodoacetamide, acryl, acylamino, mixed anhydrides, azide, acid chloride, acid bromide, hydroxide, sulfonyl chloride, vinyl sulfone, carbodiimide, maleimide or diazo.

7. Compounds according to claim 6, in which the activated carboxyl group is selected from

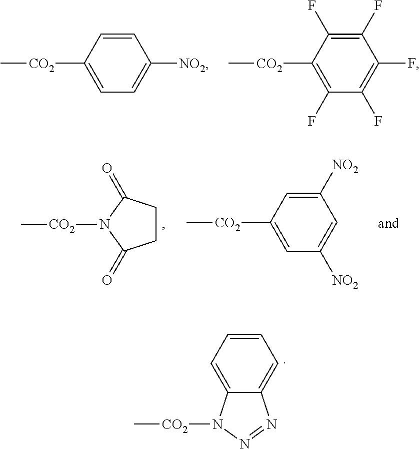

8. Use of compounds of general formula VIIa or VIIb according to claim 1 for the production of conjugates with a biomolecule.

9. Use according to claim 8, in which the biomolecule is selected from the group that consists of:
Biopolymers, proteins, such as proteins that have a biological function, HSA, BSA, etc., proteins and peptides, which accumulate at certain spots in the organism (e.g., in receptors, cell membranes, ducts, etc.), peptides that can be cleaved by proteases, peptides with predetermined synthetic sites of rupture (e.g., labile esters, amides, etc.), peptides that are cleaved by metalloproteases, peptides with photocleavable linkers, peptides with oxidative agents (oxydases) and cleavable groups, peptides with natural and unnatural amino acids, glycoproteins (glycopeptides), signal proteins, antiviral proteins and apoctosis, synthetically modified biopolymers such as biopolymers that are derivatized with linkers, modified metalloproteases and derivatized oxydase, etc., carbohydrates (mono- to polysaccharides), such as derivatized sugars, sugars that can be cleaved in the organism, cyclodextrins and derivatives thereof, amino sugars, chitosan, polysulfates and acetylneuraminic acid derivatives, antibodies, such as monoclonal antibodies, antibody fragments, polyclonal antibodies, minibodies, single chains (also those that are linked by linkers to multiple fragments), red blood corpuscles and other blood components, cancer markers (e.g., CAA) and cell adhesion substances (e.g., Lewis X and anti-Lewis X derivatives), DNA and RNA fragments, such as derivatized DNAs and RNAs (e.g., those that were found by the SELEX process), synthetic RNA and DNA (also with unnatural bases), PNAs (Hoechst) and antisense, β-amino acids (Seebach), vector amines for transfer into the cell, biogenic amines, pharmaceutical agents, oncological preparations, synthetic polymers, which are directed to a biological target (e.g., receptor), steroids (natural and modified), prostaglandins, taxol and derivatives thereof, endothelins, alkaloids, folic acid and derivatives thereof, bioactive lipids, fats, fatty acid esters, synthetically modified mono-, di- and triglycerides, liposomes, which are derivatized on the surface, micelles that consist of natural fatty acids or perfluoroalkyl compounds, porphyrins, texaphrines, expanded porphyrins, cytochromes, inhibitors, neuramidases, neuropeptides, immunomodulators, such as FK 506, CAPE and gliotoxin, endoglycosidases, substrates that are activated by enzymes such as calmodulin kinase, casein-kinase II, glutathione-S-transferase, heparinase, matrix-metalloproteases, β-insulin-receptor-kinase, UDP-galactose 4-epimerase, fucosidases, G-proteins, galactosidases, glycosidases, glycosyltransferases and xylosidase, antibiotics, vitamins and vitamin analogs, hormones, DNA intercalators, nucleosides, nucleotides, lectins, vitamin B12, Lewis-X and related substances, psoralens, dienetriene antibiotics, carbacyclins, VEGF (vascular endothelial growth factor), somatostatin and derivatives thereof, biotin derivatives, antihormones, tumor-specific proteins and synthetic agents, polymers that accumulate in acidic or basic areas of the body (pH-controlled dispersion), myoglobins, apomyoglobins, etc., neutrotransmitter peptides, tumor necrosis factors, peptides that accumulate in inflamed tissues, blood-pool reagents, anion- and cation-transporter proteins, polyesters (e.g., lactic acid), polyamides and polyphosphates.

10. Process for the production of compounds of formulas VIIa and VIIb according to claim 1, characterized in that in a way that is known in the art, in compounds of general formulas VII'a and VII'b 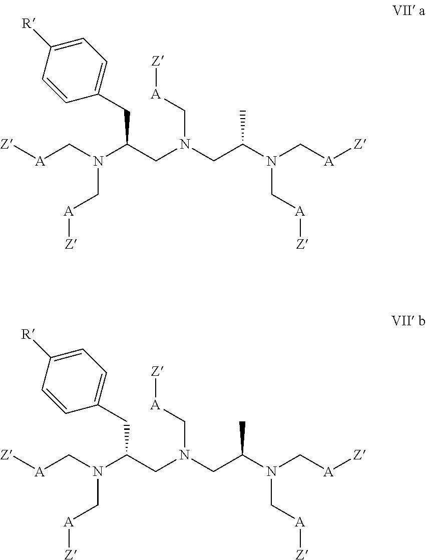
in which Z′ means a carboxyl protective group, protective groups Z′ are cleaved, and the thus obtained acids are reacted in a way that is known in the art with at least one metal oxide or metal salt of an element of atomic numbers 21-29, 31, 32, 37-39, 42-44, 46, 47, 49, 58-71, 75, 77, 82 or 83, and then, if desired, acid hydrogen atoms that are present with inorganic and/or organic acids or amino acids are converted into physiologically compatible salts.

11. Pharmaceutical agent that contains at least one physiologically compatible compound according to claim 2.

12. Pharmaceutical agent that contains at least one physiologically compatible compound according to claim 3.

13. Use of a compound according to claim 2 for the production of agents for NMR diagnosis.

14. Use of a compound according to claim 3 for the production of agents for radiodiagnosis or radiotherapy.

15. Kit for the production of radiopharmaceutical agents, comprising a compound according to claim 1, in which Z is a hydrogen atom, and a compound of a radioactive element of atomic numbers 26, 27, 29, 31, 32, 37-39, 43, 46, 61, 62, 64, 67, 70, 71, 75, 77, 82 and 83.
